(19) United States
(12) Patent Application Publication
(10) Pub. No.: US 2023/0243812 A1
(43) Pub. Date: Aug. 3, 2023

(54) ENGINEERED CARDIAC TISSUE STRUCTURES AND METHODS FOR FORMATION AND USE THEREOF

(71) Applicant: President and Fellows of Harvard College, Cambridge, MA (US)

(72) Inventors: Gilyong Lee, Boston, MA (US); Sung-Jin Park, Johns Creek, GA (US); Kevin Kit Parker, Cambridge, MA (US); Andre G. Kleber, Boston, MA (US)

(21) Appl. No.: 18/096,629

(22) Filed: Jan. 13, 2023

Related U.S. Application Data
(60) Provisional application No. 63/299,920, filed on Jan. 15, 2022.

(51) Int. Cl. G01N 33/50

(52) U.S. Cl. CPC G01N 33/5088; G01N 33/5061

(57) ABSTRACT

Engineered cardiac tissue structures and methods for forming the engineered cardiac tissue structures are provided herein. An example engineered cardiac tissue structure includes a support having a first side and a first engineered cardiac tissue layer disposed at the first side of the support. The first engineered cardiac tissue layer can include a geometrically insulated cardiac tissue node. Another example engineered cardiac tissue structure includes a support, a first engineered cardiac tissue layer, and a second engineered cardiac tissue layer. Excitation and contraction of the first engineered cardiac tissue layer bends the support and strains the second engineered cardiac tissue layer inducing excitation and contraction of the second engineered cardiac tissue layer. The excitation and contraction of the second engineered cardiac tissue layer bends the support and strains the first engineered cardiac tissue layer inducing excitation and contraction of the first engineered cardiac tissue layer thereby producing antagonistic cyclic contractions.

### RELATED APPLICATIONS

This application claims the benefit of priority to U.S. Provisional Application No. 63/299,920, filed on Jan. 15, 2022, the entire contents of which are incorporated herein by reference.

### GOVERNMENT SUPPORT

This invention was made with government support under TR000522 awarded by the National Institutes of Health (NIH), and under 2011754 awarded by the National Science Foundation (NSF). The government has certain rights in the invention.

### TECHNICAL FIELD

Embodiments of the disclosure relate to engineered cardiac tissue structures, methods for forming the engineered cardiac tissue structures, and methods of use for the engineered cardiac tissue structures.

### BACKGROUND OF THE INVENTION

The current need for organ and tissue replacement, tissue repair and regeneration for patients is continually growing such that supply is not meeting the high demand primarily due to a paucity of donors as well as biocompatibility issues that lead to immune rejection of the transplant. In an effort to overcome these drawbacks, scientists working in the field of tissue engineering and regenerative medicine have investigated the use of scaffolds as an alternative to transplantation. In addition to use for tissue replacement, tissue repair and regeneration, engineered tissues that recapitulate biological tissues are also useful for in vitro testing of pharmacological agents and for in vitro disease models. However, creating fully matured and functional cardiac tissue in vitro is still challenging. For example, current engineered tissues or organs lack of capabilities of autonomous homeostatic regulation and adaptive behavioral control (e.g., autonomous self-pacing and independent motion control).

Accordingly, there is a need for improved methods and engineered tissue structures for creating autonomous systems capable of homeostatic regulation and adaptive behavioral control and providing a more granular analysis of structure-function relationship in cardiovascular physiology.

### SUMMARY OF THE INVENTION

Some embodiments of the present invention include engineered cardiac tissue structures (e.g., engineered cardiac tissue structures having geometrically insulated cardiac tissue nodes and multilayer engineered cardiac tissue structures) and methods for forming the engineered cardiac tissue structures.

In a first aspect, the present invention provides an engineered cardiac tissue structure that includes a support having a first side and a first engineered cardiac tissue layer disposed at the first side of the support. The first engineered cardiac tissue layer includes a first plurality of cardiomyocytes. The first engineered cardiac tissue layer further includes a first region having a first area, a second region having a second area smaller than the first area, and a third region forming a conduction pathway for propagation of action potentials between the second region and the first region. The second region forms a geometrically insulated cardiac tissue node.

In one embodiment, a minimum width of the third region, as measured perpendicular to the conduction pathway, is less than a maximum width of the second region.

In one embodiment, more cardiomyocytes of the first plurality of cardiomyocytes are in the first region than in the second region.

In one embodiment, the first region, the second region and the third region collectively form a cardiac cell region having a cardiac cell region perimeter, and wherein the first plurality of cardiomyocytes do not contact any cardiac cells beyond the cardiac cell region perimeter.

In one embodiment, the cardiac cell region perimeter around the first region has one or more corners, and the cardiac cell region perimeter is rounded at each of the one or more corners.

In one embodiment, the first region, the second region, and the third region are sized and shaped for spontaneous contraction of the first engineered cardiac tissue layer to initiate in the second region more often than in the first region.

In one embodiment, the first region, second region, and third region are sized and shaped for a higher probability of initiation of spontaneous contraction of the first engineered cardiac tissue layer in the second region than in the first region.

In one embodiment, the support comprises a hydrogel layer.

In one embodiment, the hydrogel layer comprises gelatin.

In one embodiment, the second region acts as a cardiac pacemaker for the engineered cardiac tissue structure.

In one embodiment, the engineered cardiac tissue structure further includes a second engineered cardiac tissue layer disposed at a second side of the support opposite the first side of the support. The second engineered cardiac tissue layer has an anisotropic tissue orientation and includes a second plurality of cardiomyocytes. The first engineered cardiac tissue layer is physically separated from the second engineered cardiac tissue layer by a thickness of the support.

In one embodiment, excitation and contraction of the first engineered cardiac tissue layer bends the support and strains the second engineered cardiac tissue layer inducing excitation and contraction of the second engineered cardiac tissue layer. The excitation and contraction of the second engineered cardiac tissue layer bends the support and strains the first engineered cardiac tissue layer inducing excitation and contraction of the first engineered cardiac tissue layer thereby producing antagonistic cyclic contractions.

In one embodiment, the antagonistic cyclic contractions are self-sustaining cyclic contractions.

In one embodiment, the antagonistic cyclic contractions are spontaneous, self-sustaining cyclic contractions.

In one embodiment, a self-propelled swimming structure includes the engineered cardiac tissue structure of any one of the above described embodiments of the first aspect, a front body portion coupled to or attached to a first end of the engineered cardiac tissue structure, and a rear body portion coupled to or attached to a second end of the engineered cardiac tissue structure.

In one embodiment, a young's modulus of the front body portion is greater than a young's modulus of the support. A young's modulus of the rear body portion is greater than the young's modulus of the support.

In one embodiment, the self-propelled swimming structure further includes at least one buoyancy element attached to or coupled to the front body portion, the rear body portion, or both. The at least one buoyancy element is selected for the self-propelled swimming structure to maintain neutral buoyancy in a culture medium.

In one embodiment, the at least one buoyancy element is shaped to maintain directional stability of the self-propelled swimming structure.

In a second aspect, the present invention provides an engineered cardiac tissue structure that includes a support, a first engineered cardiac tissue layer, and a second engineered cardiac tissue layer. The support has a first side and a second side opposite the first side, the first side being patterned to promote anisotropic tissue formation, and the second side being patterned to promote anisotropic tissue formation. The first engineered cardiac tissue layer is disposed at the first side of the support. The first engineered cardiac tissue layer has an anisotropic tissue orientation and includes a first plurality of cardiomyocytes. The second engineered cardiac tissue layer is disposed at the second side of the support. The second engineered cardiac tissue layer has an anisotropic tissue orientation and includes a second plurality of cardiomyocytes. The first engineered cardiac tissue layer is physically separated from the second engineered cardiac tissue layer by a thickness of the support. Excitation and contraction of the first engineered cardiac tissue layer bends the support and strains the second engineered cardiac tissue layer inducing excitation and contraction of the second engineered cardiac tissue layer. The excitation and contraction of the second engineered cardiac tissue layer bends the support and strains the first engineered cardiac tissue layer inducing excitation and contraction of the first engineered cardiac tissue layer thereby producing antagonistic cyclic contractions.

In one embodiment, the antagonistic cyclic contractions are self-sustaining.

In one embodiment, the antagonistic cyclic contractions are spontaneous.

In one embodiment, the support has a bending stress in a range of 5 kPa to 15 kPa.

In one embodiment, the support comprises a hydrogel layer.

In one embodiment, the hydrogel layer includes gelatin.

In one embodiment, the support has a thickness in a range of 100 microns (μm) to 300 μm.

In one embodiment, the first engineered cardiac tissue layer includes a first region having a first area, a second region having a second area smaller than the first area, and a third region forming a conduction pathway for propagation of action potentials between the second region and the first region. The second region forms a geometrically insulated cardiac tissue node.

In one embodiment, a self-propelled swimming structure includes the engineered cardiac tissue structure of any one of the above described embodiments of the second aspect, a front body portion coupled to or attached to a first end of the engineered cardiac tissue structure, and a rear body portion coupled to or attached to a second end of the engineered cardiac tissue structure.

In one embodiment, a young's modulus of the front body portion is greater than a young's modulus of the support. A young's modulus of the rear body portion is greater than the young's modulus of the support.

In one embodiment, the self-propelled swimming structure further includes at least one buoyancy element attached to or coupled to the front body portion, the rear body portion, or both. The at least one buoyancy element is selected for the self-propelled swimming structure to maintain neutral buoyancy in a culture medium.

In one embodiment, the at least one buoyancy element is shaped to maintain directional stability of the self-propelled swimming structure.

In a third aspect, the present invention provides a method of forming a functional cardiac tissue structure. The method includes providing or obtaining a support having a first side and a second side opposite the first side, the first side being patterned and configured to promote anisotropic tissue formation, and the second side being patterned and configured to promote anisotropic tissue formation. The method further includes seeding the first side of the support and the second side of the support with cardiomyocytes, and growing a first cardiac tissue layer on the first side of the support and growing a second cardiac tissue layer on the second side of the support, the first cardiac tissue layer physically separated from the second cardiac tissue layer by a thickness of the support, thereby forming a functional cardiac tissue structure.

In one embodiment, during growth of the first cardiac tissue layer on the first side of the support and growth of the second cardiac tissue layer on the second side of the support, excitation and contraction of the first cardiac tissue layer bends the support and strains the second cardiac tissue layer inducing excitation and contraction of the second cardiac tissue layer, and the excitation and contraction of the second cardiac tissue layer bends the support and strains the first cardiac tissue layer inducing excitation and contraction of the first cardiac tissue layer producing antagonistic cyclic contractions.

In one embodiment, the antagonistic cyclic contractions are self-sustaining cyclic contractions.

In one embodiment, the antagonistic cyclic contractions are spontaneous, self-sustaining cyclic contractions.

In another aspect, the present invention provides a method for identifying a compound that modulates a cardiac tissue function. The method includes providing an engineered cardiac tissue structure as described herein. The method also includes contacting the cardiac tissue structure with a test compound, and determining the effect of the test compound on a cardiac tissue function in the presence and absence of the test compound. A modulation of the cardiac tissue function in the presence of the test compound as compared to the cardiac tissue function in the absence of the compound indicates that the test compound modulates the cardiac tissue function, thereby identifying a compound that modulates a cardiac tissue function.

In another aspect, the present invention provides a method for identifying a compound useful for treating or preventing a cardiac tissue disease. The method includes providing an engineered cardiac tissue structure as described herein. The method also includes contacting the cardiac tissue structure with a test compound. The methods also includes determining the effect of the test compound on a cardiac tissue function in the presence and absence of the test compound. A modulation of the cardiac tissue function in the presence of the test compound as compared to the cardiac tissue function in the absence of the test compound indicates that the test compound modulates the cardiac tissue function, thereby identifying a compound useful for treating or preventing a cardiac tissue disease.

In some embodiments, the cardiac tissue function is a biomechanical activity. In some embodiments, the biomechanical activity is one or more of contractility, cell stress, cell swelling, and rigidity. In some embodiments, the biomechanical activity is one or more of stem cell activation, stem cell maturation, tissue morphogenesis, and tissue remodeling.

In some embodiments, the tissue function is an electrophysiological activity. In some embodiments, the electrophysiological activity is a voltage parameter selected from the group consisting of action potential, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release.

Other features and advantages of the invention will be apparent from the following detailed description and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically depicts a perspective view of an engineered tissue structure having a geometrically insulated tissue node in accordance with some embodiments.

FIG. 2 schematically depicts various engineered tissue structures having geometrically insulated tissue nodes in accordance with some embodiments.

FIG. 3A schematically depicts a cross-sectional view of a multi-layer engineered tissue structure in accordance with some embodiments.

FIG. 3B schematically depicts an exploded view of the multi-layer engineered tissue structure in accordance with some embodiments.

FIG. 4A schematically depicts an exploded view of an example multi-layer engineered tissue structure having a geometrically insulated tissue node in accordance with some embodiments.

FIG. 4B schematically depicts an exploded view of another example multi-layer engineered tissue structure having a geometrically insulated tissue node in accordance with some embodiments.

FIG. 5A schematically depicts aspects of a heart incorporated into design of a biohybrid fish in the Example including intrinsic autonomous muscle control of a heart with mechano-electrical signaling adaptively responding to dynamic mechanical pressures in heart cells by inducing changes in electrophysiology via stretch-activated mechanosensitive proteins, and automaticity of cardiac sinoatrial node that is structurally and functionally insulated from the surrounding myocardium and initiates spontaneous electrical activity.

FIG. 5B schematically depicts a muscular bilayer in a biohybrid fish in which the shortening of contracting muscles on each side directly translates to axial stretching of the opposite side muscle leading to stretch-induced antagonistic muscle contractions in accordance with some embodiments.

FIG. 5C schematically depicts a G-node including functionally isolated cardiomyocytes (CMs) that generate spontaneous muscle activation rhythms in accordance with some embodiments.

FIG. 5D schematically depicts an autonomously swimming biohybrid fish equipped with the muscular bilayer of FIG. 5B and the G-node of FIG. 5C in accordance with some embodiments.

FIG. 6A is an image of the biohybrid fish made of human stem cell derived CMs in the Example.

FIG. 6B depicts mesostructure of the muscular bilayer structure of the biohybrid fish of FIG. 6A in which a gelatin posterior body was sandwiched by two muscle tissues expressing either a blue light-sensitive opsin, ChR2 (green) or a red light-sensitive opsin, ChrimsonR (red).

FIG. 6C includes images of a microstructure of the muscular bilayer tissue with representative immunostaining images of both tissues (sarcomeric a-actinin, gray, and nuclei, blue) showing that Z-lines of the sarcomeres (the cell force-generating units) are perpendicular to antero-posterior axis.

FIG. 6D schematically depicts an exploded view of the biohybrid fish including five layers of body architecture with a symmetrical body across a left-right plane but with asymmetrical body along both antero-posterior and dorso-ventral axis was designed to maintain directional body stability against roll and propel the body forward in accordance with some embodiments.

FIG. 7A schematically depicts alternating blue and red light stimulation induced contraction of the ChR2- and ChrimsonR-expressing muscles, respectively, for optogenetically induced BCF propulsion in a biohybrid fish in Example in accordance with some embodiments.

FIG. 7B includes images of body kinematics and hydrodynamics of the biohybrid fish in the Example during a one and half tail-beat cycle during which left and right muscles work antagonistically against each other and lead to rhythmically sustained body and caudal fin (BCF) propulsion. PIV flow measurements in the images highlight the shedding of positive and negative vortex pair at every lateral tail excursion. The images correspond to i) and v) peak contraction of left muscle; ii) and vi) recovery to straight position; and iii) peak contraction of right muscle.

FIG. 7C depicts simulations of corresponding midline kinematics for the biohybrid fish (time step: 50 msec) in the Example.

FIG. 7D is a graph of kinematic analysis of seven strokes and correlation between optogenetic muscle activation and BCF locomotion in a biohybrid fish in the Example. (n=7 strokes; data represent mean±SEM).

FIG. 7E is a graph of the curvature of the midline during five consecutive left and right muscle strokes in a biohybrid fish in the Example.

FIG. 7F is a graph of the moving distance of the biohybrid fish of the Example.

FIG. 7G is a graph of the positive relationship between pacing frequency and moving speed of optogenetically stimulated biohybrid fish (n=31 videos from 7 stingrays (3), 27 videos from 6 rat fish, 54 videos from 12 human fish; data represent mean±SEM).

FIGS. 8A-8L. Autonomous BCF Propulsion. (A to G) Mechano-electrical signaling of muscular bilayer: Spontaneous activation of one-side muscle induced consecutive contraction of the opposite-side muscle through mechano-electrical signaling between muscular bilayer tissues. (A). Representative time lapse images of consecutive antagonistic muscle contraction of 49-days old biohybrid fish (B); midline kinematics (time step: 100 msec) (C); correlation between spontaneous muscle activation and the moving distance (D); curvature of the midline during five consecutive left and right muscle strokes (E). Empirical probability of antagonistic contraction (F) and moving speed (G) of self-paced biohybrid fish treated with stretch-activated channel blockers, 250 μM streptomycin (n=4 biohybrid fish) and 100 μM Gd3+ (n=5 biohybrid fish) (box plot: center line, box limits, and whiskers indicates mean, SEM, and the first and third quartiles, respectively). The treatment of stretch-activated channel blockers, streptomycin and Gd3+, reduced the chance of antagonistic muscle contraction as well as swimming speed of biohybrid fish. (H to L) Geometrically insulated node (G-node), Activation pattern of biohybrid fish without G-node (H) and with G-node (I); Probability of muscle activation sites (J). Spontaneous muscle activation from G-node dominates spontaneous activation from the corners (n=6 biologically independent samples w/o G-node and 5 samples w/ G-node). Tail-beat frequency of biohybrid fish equipped with single-layer (n=9 videos from 9 fish), muscular bilayer (n=20 videos from 14 fish), and muscular bilayer with G-node (n=18 videos from 5 fish) (K). Significance was determined by Tukey-Kramer honestly significant difference test. Positive relationship between pacing frequency and moving speed of autonomously swimming biohybrid fish (L) (n=30 videos from 19 autonomously swimming biohybrid fish and 54 videos from optogenetically swimming biohybrid fish). Data represent mean±SEM.

FIGS. 9A-9H. Comparison of Swimming Performance Between Biohybrid and Aquatic Swimmers. (A and B) Comparison of swimming performance in biohybrid walkers and swimmers: locomotion speed (A) and speed per unit muscle mass (B). (C to F) PIV analysis of biohybrid fish (body length, lb: 14 mm) (C); wild-type juvenile zebrafish (lb: 12 mm) (D); white molly (lb: 19 mm) (E); and Micro-devario kubotai (lb: 20 mm) (F). (G and H) The scaling analysis of biohybrid fish and wild-type swimmers with Re-St (G) and Sw-Re (H) (n=30 videos from 19 biohybrid fish).

FIGS. 10A-10C. Long-Term Swimming Performance Analysis. (A and B) Trajectory (Grids, 1 cm) (A) and corresponding tail-beat angle (B) of 108-days old biohybrid fish with 79% antagonistic contractions. (C) Swimming performance for 108 days (n=4 fish). Biohybrid fish equipped with muscular bilayer demonstrated enhanced contracting amplitude, maximum swimming speed, and muscle coordination for the first month and maintained their performance at least 108 days, while fish made with single-layer muscle decreased contracting amplitude after 28 days. (n=4 fish; data represent mean±SEM).

FIGS. 11A-11B. Optogenetic Cardiac Tissue Engineering. (A) Construct design of lentiviral vector for cardiac-specific transduction of channelrhodopsin-2 (ChR2) and ChrimsonR. The lentiviral vectors for ChR2 and ChrimsonR transduction were constructed with a cardiac-specific promotor, cTnT promoter, and a fluorescence tag, eGYP or mCherry. (B) Immunofluorescence images show that ChR2 and ChrimsonR were mutually exclusively expressed in each muscle layers (representative of n=10 recorded images). The muscular structure of muscular bilayer was aligned along the longitudinal direction of the micro-mold gelatin substrate.

FIGS. 12A-12D. Optogenetic Control for Muscular Bilayer Cantilever Construct at A Wide Pacing Range: (A) 1 Hz, (B) 1.5 Hz, (C) 2.5 Hz, and (D) 3 Hz. Blue and red light independently activated the ChR2- and the ChrimsonR-expressed muscle layers, respectively. At low pacing frequencies (1 and 1.5 Hz), the contraction and relaxation between muscle on one side and muscle on the other side are fully decoupled. Thus, the relaxation of each muscle layer causes the muscular bilayer cantilever to recover from the maximum bending to the near-straight position. However, as pacing frequencies increase (2.5 and 3 Hz), the relaxation of muscle on one side overlapped with the consecutive contraction of the muscle on the other side. Thus, the consecutive muscle contraction dramatically increases the recovering speed of muscular bilayer tissue at the near-straight position. Data are representative of three muscular bilayer tissues.

FIGS. 13A-13J. Fabrication Process for Biohybrid Fish. (A) Two pieces of painter's tape were attached together on their adhesive side. (B) The biohybrid fish outline was then laser cut onto the painter's tape. (C to E) A microbial transglutaminase (MTG) and gelatin mixture was sandwiched between two polydimethylsiloxane (PDMS) stamps with line groove features to make a micropatterned gelatin body parallel to the biohybrid fish's longitudinal axis (C and D). The gelatin portions were cured and crosslinked overnight, then detached from the PDMS stamps (E). (F) Both sides of gelatin portions were coated with fibronectin. (G and H) ChR2-expressing human stem cell-derived CMs were seeded onto one side at first (G), and after a day of culture, ChrimsonR-expressing CMs were seeded onto the other side (H). (I) The Biohybrid fish were released from the excess painter's tape after 3 days in culture. (J) Lastly, a plastic floater fin was inserted into the tissue-engineered fish.

FIGS. 14A-14E. Design of Biohybrid Fish. (A) without G-node, (B) with G-node, (C) with long body length, and (D) with small point node design G-node, and (E) design of experimental setup.

FIGS. 15A-15F. Body Length and Mass of Biohybrid and Wild-Type Fish. (A) Length and thickness of posterior gelatin body (n=10 biohybrid fish; data represent mean±SD). (B) Mass composition of biohybrid fish. (C) Muscle mass of biohybrid fish. (D) Mass of live larval zebrafish, white molly, and Micro-devario kubotai. (E) Comparison of ratios of mass to body length between biohybrid and wild-type fish. (F) Comparison of ratios of muscle mass to body length between biohybrid and wild-type fish. (n=7 biohybrid fish, n=3 wild-type fish; data represent mean±SD).

FIGS. 16A-16D. Body Kinematics of Biohybrid Fish Swimming Under External Optogenetic Control (Solid Line) and Internal Self-Pacing (Square Marker). (A) 1:1 stimulus-response of optogenetically paced biohybrid fish. The “1:1 stimulus to response” was quantified by measuring the response percentage of tissues contracting to each optical stimuli input. (B) Tail oscillation amplitude. (C) Tail beat angle. (D) Tail beat angular speed. Biohybrid fish maintained both tail oscillation amplitude and tail beat angle even at high pacing frequencies (3 to 4 Hz). Thus, an increase in the pacing frequency led to an increase in the tail beat angular speed of both external optogenetic controlled and autonomously swimming biohybrid fish, which improved their swimming speed. (n=30 videos from 19 spontaneously paced fish and 54 videos from 12 optogenetically paced fish; data represent mean±SEM).

FIG. 17. NRVM-Based Biohybrid Fish Accelerating Speed By Increasing Pacing Frequency. The NRVM-based biohybrid fish was stimulated with optical pacing with varying pacing frequencies (1, 1.5, 2, 2.5 and 3 Hz). Blue and red light independently activate ChR2- and ChrimsonR-expressed muscle layers, respectively, with 180 degree-phase shifts between red and blue lights. The data were acquired by video tracking. The data are representative of independent experiments of 6 rat fish with similar results.

FIGS. 18A-18B. Disruption of antagonistic muscle contraction caused by the treatment of stretch-activated channel blockers, streptomycin (A) and Gd³⁺ (B). The larger tail-beat angle (>110 degrees) induced antagonistic muscle contraction, but both stretch-activated ion channels inhibitors significantly decreased the empirical probability of antagonistic contractions in the biohybrid fish (Comparisons for all pairs using Tukey-Kramer Honest Significant Difference, n=3 streptomycin-treated fish; 5 Gd³⁺-treated fish).

FIGS. 19A-F. Body Kinematics of Biohybrid Fish Before and After Gd³⁺ Treatment. (A and B) Tail-beat angle (A) and its frequency-domain analysis (power spectral density) (B) of husk-CMs-based biohybrid fish before Gd³⁺ treatment. (C and D) Tail-beat angle (C) and its frequency-domain analysis (D) of the same biohybrid fish of (A and B) after Gd³⁺ treatment. (E and F) Tail-beat angle (E) and its frequency-domain analysis (F) of another husk-CMs-based biohybrid fish after Gd³⁺ treatment. Before the inhibition of stretch-activated ion channels (Gd³⁺), the left and right muscle tissues were antagonistically contracting with the same pacing frequency (3.52 Hz in B). However, the Gd³⁺ treatment disrupted antagonistic contraction of muscular bilayer tissues: the left and right muscle tissues were independently contracting with two different pacing frequencies (2.34 and 0.98 Hz in D, and 1.95 and 1.17 Hz in F). Their frequencies were not harmonic, demonstrating the decoupling of spontaneous activities between muscular bilayer tissues. The tail-beat angles in A and B and C to F were calculated by video tracking of the body midlines of biohybrid fish, respectively. The images are representative of three independent experiments.

FIGS. 20A-20J. G-node. (A to C) The CMs in the four corners of the muscle tissue (A) and G-node (B: small point node design, and C: large circular node design) are geometrically isolated and surrounded with relatively small number of CMs. (D to F) Activation pattern initiated from anterior ventral corner (D top), posterior dorsal corner (D bottom) in tissues without G-node, and from G-node in tissue with G-node (E: small point node design and F: large circular node design). The data in (D) to (F) were acquired by video tracking, respectively. (G to J) Probability of muscle activation sites in biohybrid fish: overall comparison (G) and individual sample results without G-node (n=6 fish) (H) and with G-node (I: small point node design: n=3 fish, J: large circular node design: n=5 fish). G-node, competing with the other four corners of the muscle tissue, predominantly initiated the sequential activation waves in the tissues equipped with large circular node design. The activation waves were predominantly initiated from the anterior having corners with acute angles, and the probability of muscle activation sites from anterior sides increased up to 97% by the G-node integration. The images are representative of independent experiments.

FIG. 21. Muscle Activation in G-Node-Integrated Tissues. Design (top), Ca²⁺ propagation signals (middle), and activation map (bottom). G-node integration in rectangular tissue with sharp and rounded corners. G-nodes integrated into a rectangular tissue predominantly activated the muscle construct compared to the four corners of the muscle tissue. Furthermore, the rounded corners in the sink decreased the empirical probability of initial activation at the corners in the sink, and thus the activation probability of the integrated G-node in the rounded tissue increased to 91%.

FIG. 22. Muscle Activation In G-Node-Integrated Tissues. Design (top), Ca²⁺ propagation signals (middle), and activation map (bottom). Different shapes of G-node in rounded rectangular tissue. The square and diamond G-nodes had a similar probability of activation at the G-node to the circular design (83 and 87% vs. 91%). This indicates that acute angles in the small source tissue like the G-node are not critical in determining the activation site. Rather, it is the fewer number of cells in the smaller G-node tissue that acts as a source to initiate muscle activation.

FIG. 23. The Contribution Of The Corner Designs In The Rectangular Tissue To The Probability Of Initial Activation At The Corner. The reduced sink load caused by the decreased number of downstream cells increases ζ (the probability of initial activation at the corner compared to the probability at the G-node). For example, a rounded corner increases the number of downstream cells by 56% compared to a 90 degree shape corner, and decreases ζ by 73%, while the acute corner of the biohybrid fish body decreases the number of downstream cells by 16% compared to a 90 degree shape corner, thus increasing ζ by 220%. These results confirmed the source-sink mismatch principle that affects the initial muscle activation site. These experimental results and fitting plot can be used to guide future designs for autonomously actuating cardiac muscle systems.

FIGS. 24A-24B. Change in Midline Kinematics During the Swimming Mode Shift from Undulatory Wave Propagation (0.1 To 0.2 S) to Global Muscle Contraction (0.24 to 0.32 S). (A and B) Representative time lapse images and midline kinematics of biohybrid fish swimming via undulatory locomotion (A) and oscillatory locomotion (B). (C) Midline curvature. Biohybrid fish accelerating from rest induced sequential local muscle activation and contraction, leading to undulatory locomotion, while in subsequent muscle contractions, the biohybrid fish predominantly exhibited simultaneous global contractions and as a result, oscillatory locomotion with minimal body wave propagations. The midline curvatures were calculated by video tracking of the body midlines of biohybrid fish.

FIG. 25. Change in midline kinematics (A) and midline curvature (B) of biohybrid fish during the shift from small oscillation amplitude swimming (0 to 1 s) to large oscillation amplitude swimming (1.4 to 2.4 s). The midline curvatures were calculated by video tracking of the body midlines of biohybrid fish.

FIGS. 26A-26C. Comparison of swimming performance between the biohybrid fish before and after high tail-beat oscillation (A to C) and before and after the treatment of a stretch activated ion channel blocker, Gd³⁺ (D to F).

FIG. 27. Geometric relationship of the curvature (κ), radius of curvature (r), length of muscle tissue (l_{muscle}), and tail-beat angle (θ) (κ=1/r=θ/l_{muscle}).

### DETAILED DESCRIPTION

In the following description, it is understood that terms such as “top,” “bottom,” “middle,” “outward,” “inward,” and the like are words of convenience and are not to be construed as limiting terms.

As used herein, excitable cells refer to cells that respond to an electrical stimulation by propagating an action potential (e.g., muscle cells, neurons).

As used herein, activation of excitable cells, with respect to muscle cells, refers to contraction of the muscle cells and can also refer to propagation of an action potential (e.g., as measured by channel opening in the muscle cells). Activation of excitable cells, with respect to neurons, refers to propagation of an action potential (e.g., as measured by channel opening in the neurons).

Reference is made in detail to embodiments of the disclosure, which are illustrated in the accompanying figures and examples. Referring to the drawings in general, it will be understood that the illustrations are for the purpose of describing particular embodiments of the disclosure and are not intended to limit the same.

Whenever a particular embodiment of the disclosure is said to comprise or consist of at least one element of a group and combinations thereof, it is understood that the embodiment may comprise or consist of any of the elements of the group, either individually or in combination with any of the other elements of that group.

These and other aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

Some embodiments described herein include engineered tissue structures having geometrically insulated tissue nodes. For example, an engineered cardiac tissue structure can be a tissue structure having one or more features of heart, e.g., mechano-electrical signaling and automaticity, autonomously actuating cardiac muscular features, features of tissue-level cardiac biophysics, and/or features associated with cardiovascular physiology. An engineered cardiac tissue structure includes a support (e.g., a hydrogel layer) having a first side and a first engineered cardiac tissue layer disposed at the first side of the support. The first engineered cardiac tissue layer includes a first plurality of cardiomyocytes. In some embodiments, the first engineered cardiac tissue layer further includes a first region having a first area, a second region having a second area smaller than the first area, and a third region forming a conduction pathway for propagation of action potentials between the second region and the first region. The second region forms a geometrically insulated cardiac tissue node (G-node).

In some embodiments, similar to the engineered cardiac tissue structure, other types of cells (e.g., neurons or skeletal muscle cells) can be used to form corresponding types of engineered tissue structures. For example, an engineered tissue structure includes a support (e.g., a hydrogel layer) having a first side and a first engineered tissue layer disposed at the first side of the support. The first engineered tissue layer includes a first plurality of neurons or skeletal muscle cells. The first engineered tissue layer further includes a first region having a first area, a second region having a second area smaller than the first area, and a third region forming a conduction pathway for propagation of action potentials between the second region and the first region. The second region forms a geometrically insulated tissue node.

Some embodiments described herein include multilayer engineered cardiac tissue structures. For example, an engineered cardiac tissue structure can include a support (e.g., a hydrogel layer), a first engineered cardiac tissue layer, and a second engineered cardiac tissue layer in some embodiments. The support has a first side and a second side opposite the first side. The first side is patterned to promote anisotropic tissue formation, and the second side is patterned to promote anisotropic tissue formation. The first engineered cardiac tissue layer is disposed at the first side of the support. The first engineered cardiac tissue layer has an anisotropic tissue orientation and includes a first plurality of cardiomyocytes. The second engineered cardiac tissue layer is disposed at the second side of the support. The second engineered cardiac tissue layer has an anisotropic tissue orientation and includes a second plurality of cells (e.g., cardiomyocytes, neurons or skeletal muscle cells). The first engineered cardiac tissue layer is physically separated from the second engineered cardiac tissue layer by a thickness of the support. Excitation and contraction of the first engineered cardiac tissue layer bends the support and strains the second engineered cardiac tissue layer inducing excitation and contraction of the second engineered cardiac tissue layer. The excitation and contraction of the second engineered cardiac tissue layer bends the support and strains the first engineered cardiac tissue layer inducing excitation and contraction of the first engineered cardiac tissue layer thereby producing antagonistic cyclic contractions.

In some embodiments, similar to the engineered cardiac tissue structure, other types of cells (e.g., neurons or skeletal muscle cells) can be used to form corresponding types of engineered tissue structures. For example, an engineered tissue structure can include a support (e.g., a hydrogel layer), a first engineered tissue layer, and a second engineered tissue layer. The support has a first side and a second side opposite the first side. The first side is patterned to promote anisotropic tissue formation, and the second side is patterned to promote anisotropic tissue formation. The first engineered tissue layer is disposed at the first side of the support. The first engineered tissue layer has an anisotropic tissue orientation and includes a first plurality of neurons or skeletal muscle cells. The second engineered tissue layer is disposed at the second side of the support. The second engineered tissue layer has an anisotropic tissue orientation and includes a second plurality of neurons or skeletal muscle cells. The first engineered tissue layer is physically separated from and mechanically coupled to the second engineered tissue layer by a thickness of the support. Excitation and contraction of the first engineered tissue layer bends the support and strains the second engineered tissue layer inducing excitation and contraction of the second engineered tissue layer. The excitation and contraction of the second engineered tissue layer bends the support and strains the first engineered tissue layer inducing excitation and contraction of the first engineered tissue layer thereby producing antagonistic cyclic contractions.

Some embodiments described herein include methods of forming functional cardiac tissue structures. A method includes providing or obtaining a support (e.g., a hydrogel layer) having a first side and a second side opposite the first side. The first side is patterned and configured to promote anisotropic tissue formation, and the second side is patterned and configured to promote anisotropic tissue formation. The method further includes seeding the first side of the support and the second side of the support with cells (e.g., cardiomyocytes, neurons or skeletal muscle cells), and growing a first cardiac tissue layer on the first side of the support and growing a second cardiac tissue layer on the second side of the support. The first cardiac tissue layer is physically separated from the second cardiac tissue layer by a thickness of the support, thereby forming a functional cardiac tissue structure.

In some embodiments, similar to forming functional cardiac tissue structures, the provided methods can form other types of engineered tissue structures using other types of cells (e.g., neurons or skeletal muscle cells). For example, a method for forming functional tissue structures includes providing or obtaining a support (e.g., a hydrogel layer) having a first side and a second side opposite the first side. The first side is patterned and configured to promote anisotropic tissue formation, and the second side is patterned and configured to promote anisotropic tissue formation. The method further includes seeding the first side of the support and the second side of the support with neurons or skeletal muscle cells, and growing a first tissue layer on the first side of the support and growing a second tissue layer on the second side of the support. The first tissue layer is physically separated from the second tissue layer by a thickness of the support.

Some embodiments described herein include methods for identifying a compound that modulates a cardiac tissue function. A method includes providing an engineered cardiac tissue structure and contacting the engineered cardiac tissue structure with a test compound. The method also includes determining the effect of the test compound on a cardiac tissue function in the presence and absence of the test compound. A modulation of the cardiac tissue function in the presence of the test compound as compared to the cardiac tissue function in the absence of the test compound indicates that the test compound modulates the cardiac tissue function, thereby identifying a compound that modulates a cardiac tissue function.

Some embodiments described herein include methods for identifying a compound that modulates a tissue function. For example, where the tissue includes neurons or skeletal muscle cells. A method includes providing an engineered tissue structure (e.g., an engineered tissue structure including neurons or skeletal muscle cells) and contacting the engineered tissue with a test compound. The method also includes determining the effect of the test compound on the tissue function in the presence and absence of the test compound. A modulation of the tissue function in the presence of the test compound as compared to the tissue function in the absence of the test compound indicates that the test compound modulates the tissue function, thereby identifying a compound that modulates a tissue function.

Some embodiments described herein include methods for identifying a compound useful for treating or preventing a cardiac tissue disease. A method for identifying a compound useful for treating or preventing a cardiac tissue includes providing an engineered cardiac tissue structure, and contacting the engineered cardiac tissue structure with a test compound. The method also includes determining the effect of the test compound on the cardiac tissue function in the presence and absence of the test compound. A modulation of the cardiac tissue function in the presence of the test compound as compared to the cardiac tissue function in the absence of the test compound indicates that the test compound modulates the cardiac tissue function, thereby identifying a compound useful for treating or preventing a cardiac tissue disease.

Some embodiments described herein include methods for identifying a compound useful for treating or preventing a tissue disease. For example, where the disease affects neurons or skeletal muscle. A method for identifying a compound useful for treating or preventing a tissue disease includes providing an engineered tissue structure (e.g., an engineered tissue structure including neurons or skeletal muscle cells) and contacting the engineered tissue structure with a test compound In some embodiments, the engineered tissue structure includes neurons or skeletal muscle cells. The method also includes determining the effect of the test compound on the tissue function in the presence and absence of the test compound. A modulation of the tissue function in the presence of the test compound as compared to the tissue function in the absence of the test compound indicates that the test compound modulates the tissue function, thereby identifying a compound useful for treating or preventing a tissue disease.

### Engineered Tissue Structures

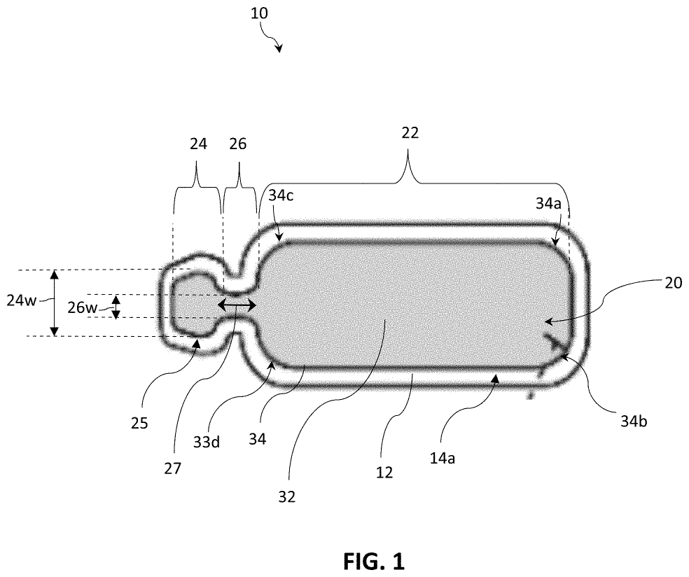

Turning to the drawings, FIG. 1 schematically depicts a perspective view of an engineered tissue structure 10 (e.g., cardiac tissue structure) having a geometrically insulated tissue node 25 in accordance with some embodiments. The engineered tissue structure 10 includes a support 12 having a first side 14a and a first engineered tissue layer 20 disposed at the first side 14a of the support 12. The first engineered tissue layer 20 includes a plurality of excitable cells. In some embodiments, this may be described as a first plurality of excitable cells. In some embodiments, the excitable cells include cardiomyocytes (CMs). In some embodiments, the excitable cells include CMs, neurons, skeletal muscle cells, or any combination of the aforementioned. The first engineered tissue layer 20 further includes a first region 22 having a first area, a second region 24 having a second area smaller than the first area, and a third region 26 forming a conduction pathway 27 for propagation of action potentials between the second region 24 and the first region 22. In some embodiments, a larger number of excitable cells are disposed in the first region 22 than in the second region 24. The first region 22, the second region 24, and the third region 26 collectively form a cell region 32 over which the plurality of excitable cells is distributed. The cell region 32 has a perimeter 34. In some embodiments, the plurality of excitable cells do not contact any cells beyond the cell region perimeter. In some embodiments, the plurality of excitable cells do not contact any excitable cells beyond the cell region perimeter.

In some embodiments, the second region 24 forms a geometrically insulated tissue node (G-node). In some embodiments, the first region 22, the second region 24, and the third region 26 are sized and shaped for initiation of spontaneous activation of excitable cells (e.g., contraction of CMs or skeletal muscle cells, propagation of APs in neurons), or initiation of a spontaneous propagating wave of activation of excitable cells, to more often occur in the second region than in the first region due to geometry of the second region 24, of the first region 22, and of the third region. In some embodiments, the first region 22, the second region 24, and the third region 26 are sized and shaped for initiation of spontaneous activation of excitable cells (e.g., contraction of CMs or skeletal muscle cells, propagation of APs in neurons), or initiation of a spontaneous propagating wave of activation of excitable cells, to have a higher probability of occurring in the second region 24 than in the first region 22. In some embodiments, the excitable cells are CMs and the second region acts as a cardiac pacemaker for the engineered tissue structure. Various factors that affect the probability of spontaneous activation (e.g. spontaneous contraction) initiating in the second region rather than the first region, or of a spontaneous propagating wave of activation initiating in the second region rather than the first region are described below and in the Example.

In some embodiments width 26w (e.g., a minimum width) of the third region 26 as measured perpendicular to the conduction pathway, is less than a width 24w (e.g., a maximum width) of the second region 24 providing increased geometric isolation for the second region 24 from the first region 22. As described below with respect to the Example, this increase geometric isolation can result in increased frequency for spontaneously initiated waves of activation and more synchronized activation, due, in part, to the reflection of intracellular currents at the tissue edges (tissue perimeter) in the smaller second region. In some embodiments, the cell region perimeter 34 around the first region 22 has corners one or more corners that are rounded, or includes only rounded corners 34a-34d. Rounding corners in the first region 22 significantly decreases the probability of spontaneous initiation of activation in the first region 22. In other embodiments, the cell region perimeter 34 around the first region 22 or the second region 24 can have at least one corner that is not rounded as further described below. As explained below with respect to the Example, whether the second region 24 has rounded corners or sharp corners may not significant affect a probability of spontaneous initiation of activation in the second region 24.

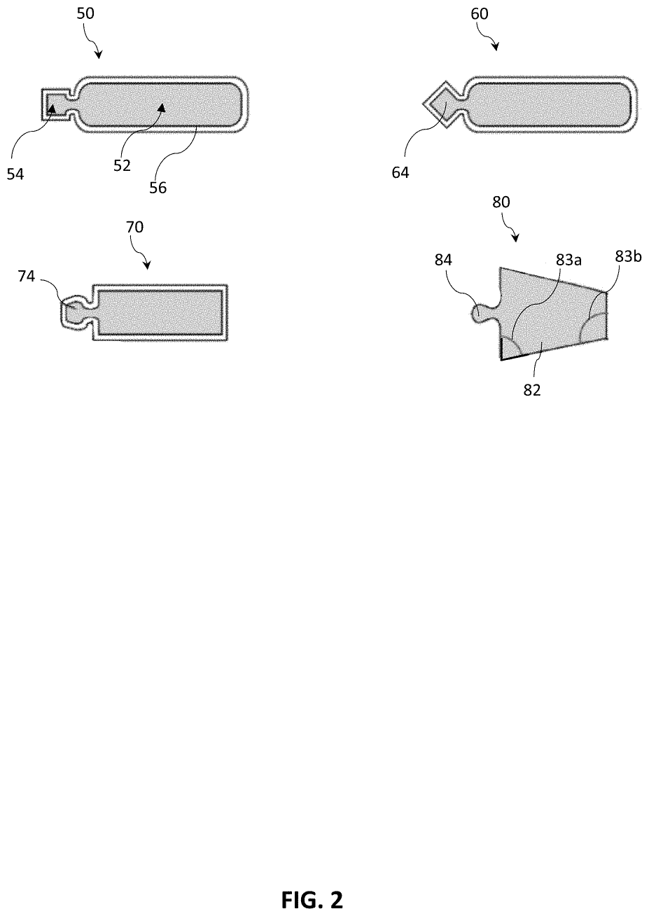

FIG. 2 schematically depicts various engineered tissue structures 50, 60, 70, 80 each having geometrically insulated tissue node in accordance with various embodiments. An engineered tissue 50 includes a first region 52 and a second region 54 having a square shape in accordance with an embodiment. A cell region perimeter 56 around the first region 52 has round corners. The cell region perimeter 56 around the second region 54 has sharp corners. An engineered tissue structure 60 includes a second region 64 having a diamond shape with sharp corners in accordance with another embodiment. An engineered tissue structure 70 includes a second region 74 that has rounded corners in accordance with another embodiment.

In engineered tissue structure 50, 60 and 70, the respective first region is rectangular in shape. In some embodiments, the first region is square in shape. In some embodiments, the first has a shape other than square or rectangular. In some embodiments, the first region may have one or more corners with internal angles different from 90 degrees. An engineered tissue structure 80 includes a first region 82 having a trapezoidal shape with sharp corners and a second region 84 having rounded corners in accordance with another embodiment. In some embodiments, a corner in the first region may have an acute internal angle of less than 90 degree 83a, and/or a corner in the second region may have an obtuse internal angle of more 90 degree 83b.

In some embodiments, a corner in the second region may have an acute internal angle of less than 90 degree 83a, and/or a corner in the second region may have an obtuse internal angle of more 90 degree 83b.

Although the respective second regions in each of engineered tissue structures 50, 60, 70 and 80 each have four corners and four sides, in some embodiments, an engineered tissue structure has more than four corners and more than four sides. In some embodiments, an engineered tissue structure has less than four corners and four sides.

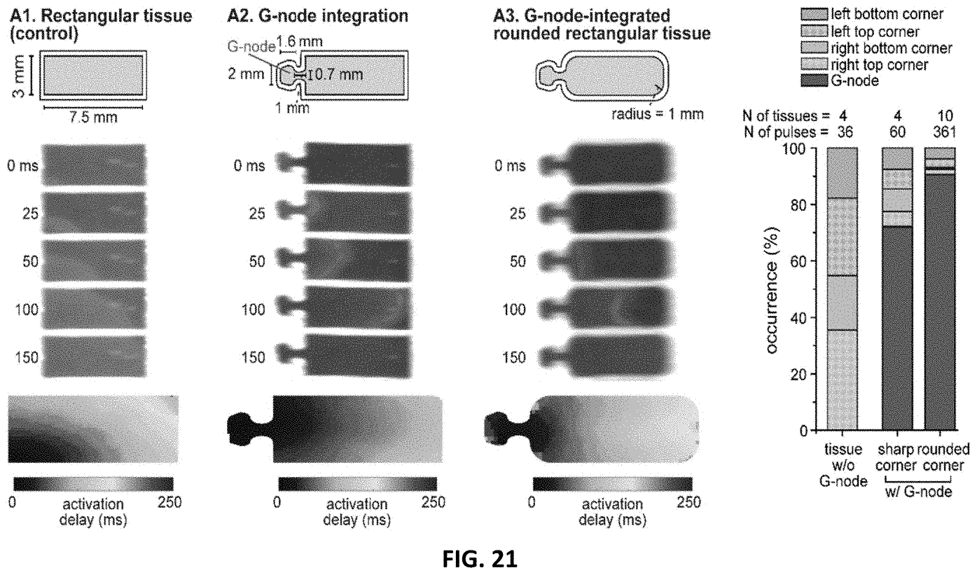

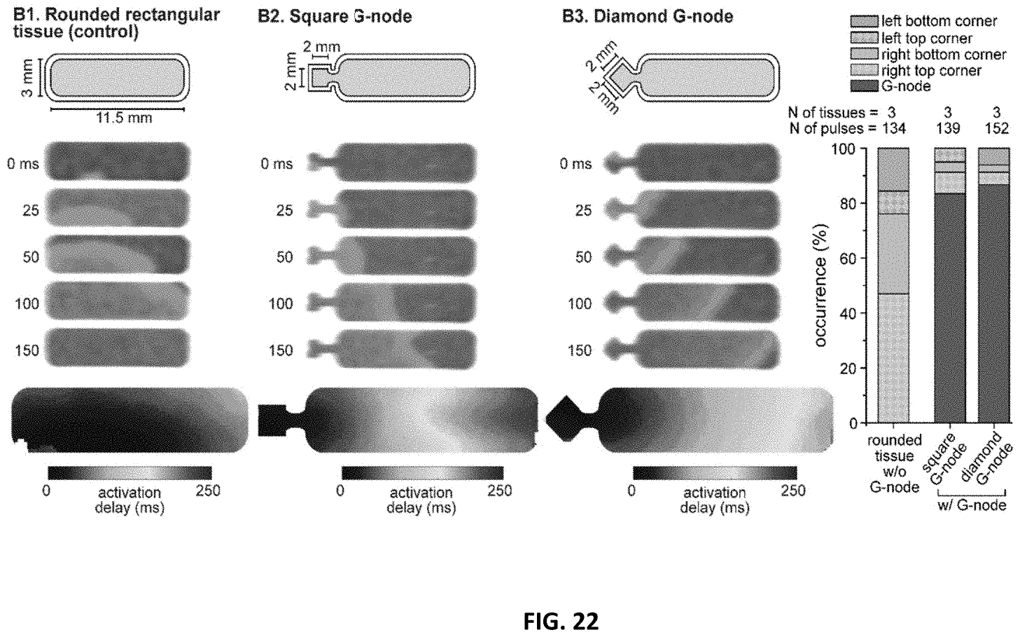

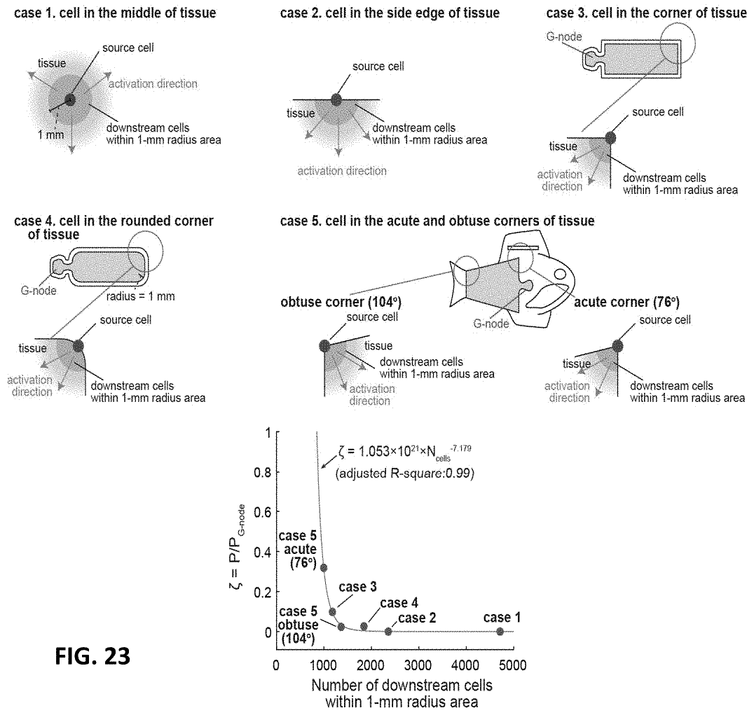

As described in the Example below with respect to FIG. 23, in an in vitro cardiac tissue model, a majority of spontaneous contractions (e.g., beats) was initiated from the perimeter of the tissue layer due to reduced source-to-load mismatch at these sites. The electrical interaction between cells in the middle of the tissue and their neighboring cells likely dampened local spontaneous activities due to source and sink mismatch (low source/high sink) and prevented tissue-level action potential formation. In contrast, spontaneous activities induced by cells at the edge sites (e.g., at the perimeter) had a higher probability of yielding tissue-level propagating action waves than the cells in the middle due to a relatively smaller number of neighboring cells (lower sink). Rounding the corners of the second section region increases a probability of spontaneous activation (e.g. spontaneous contraction) initiating in the second region rather than the first region, or of a spontaneous propagating wave of activation initiating in the second region rather than the first region. Muscle activations in G-node-integrated engineered cardiac tissue structures are described in the Example below with respect to FIGS. 21 and 22. The influence of different types of corner designs in a rectangular cardiac tissue to a probability of initial activation at a corner is described below with respect in FIG. 23.

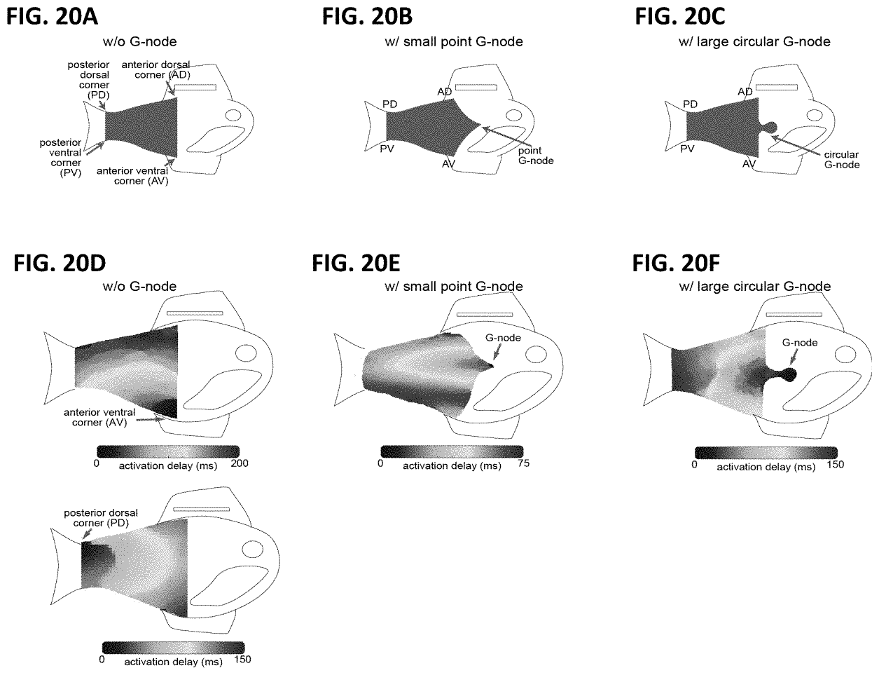

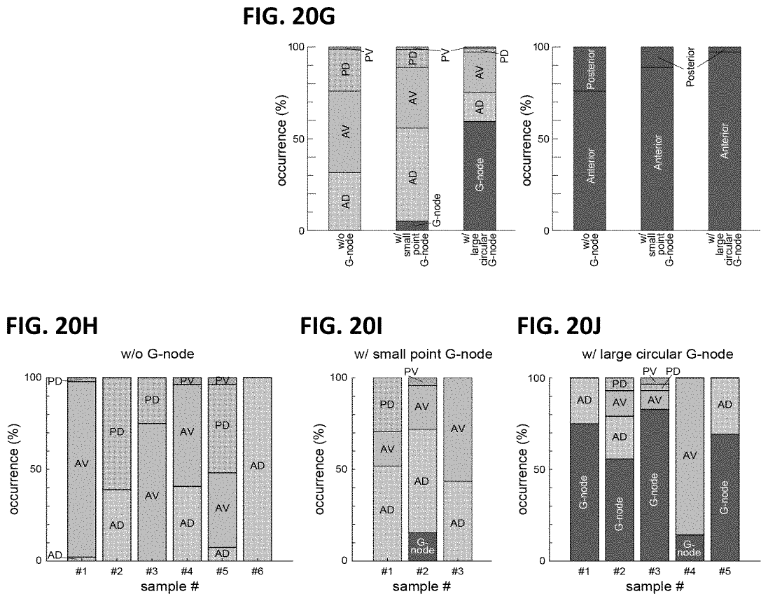

For example, as shown and described below in the Example with respect to FIGS. 20-23, the second region (e.g., G-node) predominantly spontaneously initiated muscle activation of the engineered muscle tissue structure construct relative to other four corners of the first region. Comparing two different sizes for the second region (two different G-node sizes) showed that a larger second region (G-node) containing about a group of about 1,700 cells had an increased probability of initial muscular activation in the second region (at the G-node) compared to a smaller second region (G-node) with a pointed node including about 600 cells (FIG. 20). Additionally, rounding the corners of the first region, which functions as a sink, decreased the probability of activation at the corners (FIG. 21) of the first region by increasing the number of downstream cells at each respective corner (FIG. 23). This demonstrates the significant benefit of having rounded corners in the second region. However, in contrast, the first region's corner design did not appear to significantly affect probability of activation at the first region (the G-node) (FIG. 22). This indicates that sharp corners and acute angles in the first region (the G-node) are not critical in determining whether spontaneous activation preferably occurs in the first region or the second region. Rather, this indicates that the larger perimeter to area ratio of the first region (the G-node) relative to that of the second region (the sink) synchronized electrical interaction between the geometrically distinct CMs through reflections of electronic currents and produced a relatively fast and synchronized activation over the sink tissue. One of ordinary skill in the art in view of the present disclosure will appreciate that various shapes may be employed for the first region, the second region, and the third region while still forming a G-node. Further description of aspects and features of G-nodes are provided below with respect to the Example.

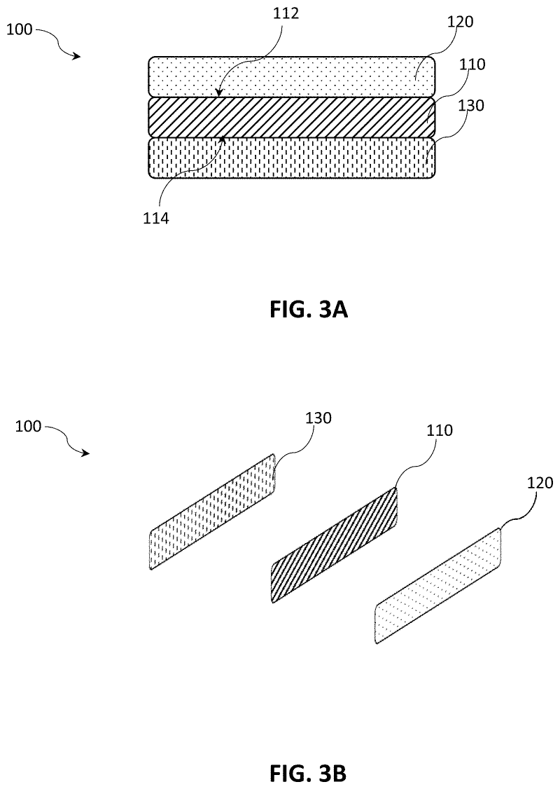

FIG. 3A schematically depicts a cross-sectional view of a multi-layer engineered tissue structure 100 in accordance with some embodiments. FIG. 3B schematically depicts an exploded view of the multi-layer engineered tissue structure 100 in accordance with some embodiments. The engineered tissue structure 100 includes a support 110, a first engineered tissue layer 120, and a second engineered tissue layer 130. The support 110 has a first side 112 and a second side 114 opposite the first side 112. The first side 112 is patterned to promote anisotropic tissue formation, and the second side 114 is patterned to promote anisotropic tissue formation. The first engineered tissue layer 120 is disposed at the first side 112 of the support 110. The first engineered tissue layer 120 has an anisotropic tissue orientation and includes a first plurality of excitable and contractile cells (e.g., cardiomyocytes or skeletal muscle cells). The second engineered tissue layer 130 is disposed at the second side 114 of the support 110. The second engineered tissue layer 130 has an anisotropic tissue orientation and includes a second plurality of excitable and contractile cells (e.g., cardiomyocytes or skeletal muscle cells). The first engineered tissue layer 120 is physically separated from the second engineered issue layer 130 by a thickness of the support 110. The multilayer engineered tissue structure is configured such that excitation and contraction of the first engineered tissue layer 120 bends the support 110 and strains the second engineered tissue layer 130 inducing excitation and contraction of the second engineered tissue layer 130. The excitation and contraction of the second engineered tissue layer 130 bends the support 110 and strains the first engineered tissue layer 120 inducing excitation and contraction of the first engineered tissue layer 120 thereby producing antagonistic cyclic contractions. In some embodiments, the antagonistic contractions are self-sustaining cyclic contractions. In some embodiments, the antagonistic cyclic contractions are spontaneous, self-sustaining cyclic contractions. Further explanation of spontaneous, self-sustaining antagonistic cyclic contractions is provided in the Example.

In some embodiments, the support 110 is or includes a hydrogel layer. In some embodiments, the hydrogel layer includes a gelatin hydrogel. In some embodiments, the thickness and stiffness or Young's modulus of the support is selected such that contraction of one of the engineered tissue layers causes sufficient tensile stress in the other engineered tissue on the opposite side of the support to produce contraction of the other layer. In some embodiments, the support has a thickness in a range of 100 μm to 300 μm. In some embodiments, the support has a bending stress in a range of 5 kPa to 15 kPa. In some embodiments, the support has a bending stress of about 10 kPa.

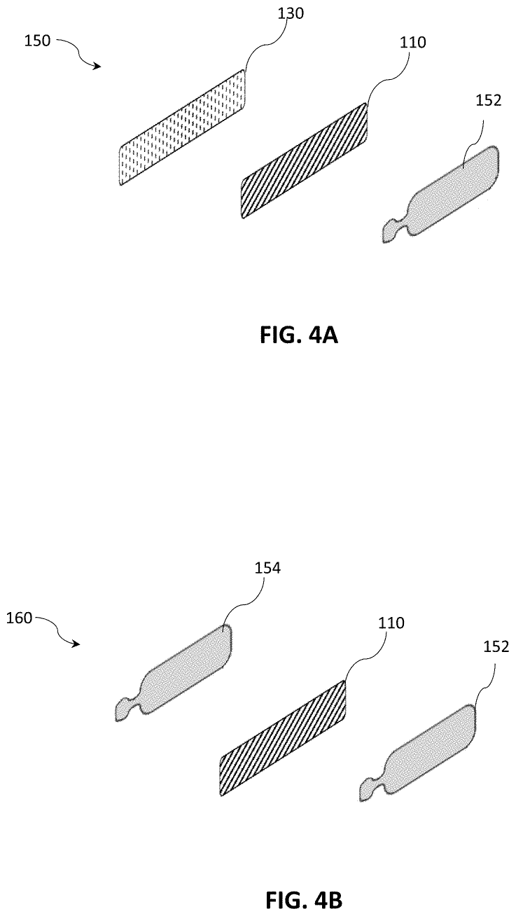

In some embodiments, at least one of the first engineered tissue layer 120 and the second engineered tissue layer 130 can have a geometrically insulated tissue node. FIG. 4A schematically depicts an exploded view of an example multi-layer engineered tissue structure 150 having a geometrically insulated tissue node in accordance with some embodiments. Compared with FIG. 3B, in the multi-layer engineered tissue structure 150 the first engineered tissue layer 120 is replaced by an engineered tissue layer 152 having a geometrically insulated tissue node. For example, in some embodiments, the first engineered tissue layer may have a shape as shown in FIG. 1 or FIG. 2.

FIG. 4B schematically depicts an exploded view of another example multi-layer engineered tissue structure 160 having a geometrically insulated tissue node on each side in accordance with some embodiments. Compared with FIG. 3B, in the multi-layer engineered tissue structure 160 the first engineered tissue layer 120 and the second engineered tissue layer 130 are replaced by engineered tissue layers 152, and 154 respectively, which each has a G-node. In some embodiments (not shown), the first engineered tissue layer 120 and the second engineered tissue layer 130 can be replaced by engineered tissue layers each having a geometrically insulated tissue node and a different configuration. For example, each engineered tissue layers can have a different size, shape, corner type or than the other engineered tissue layer, or any combination of the aforementioned.

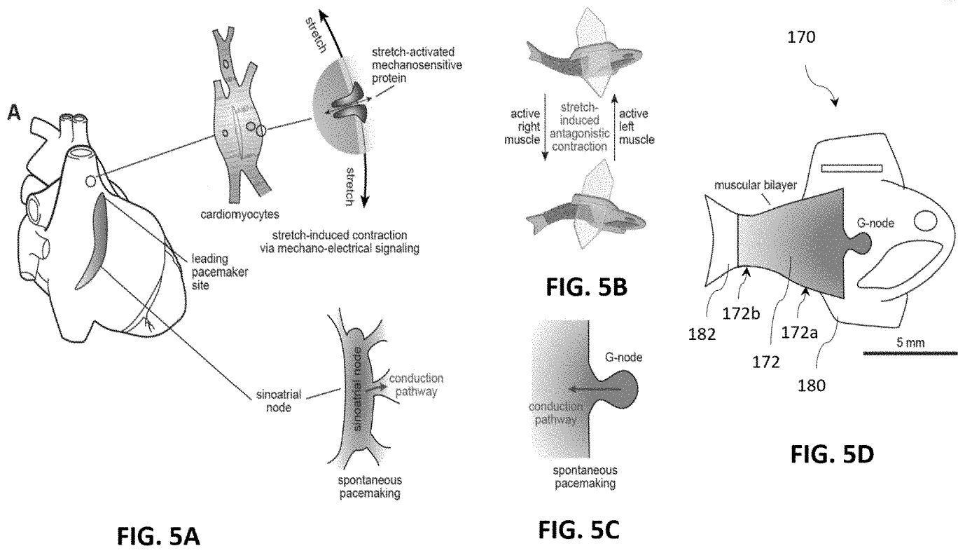

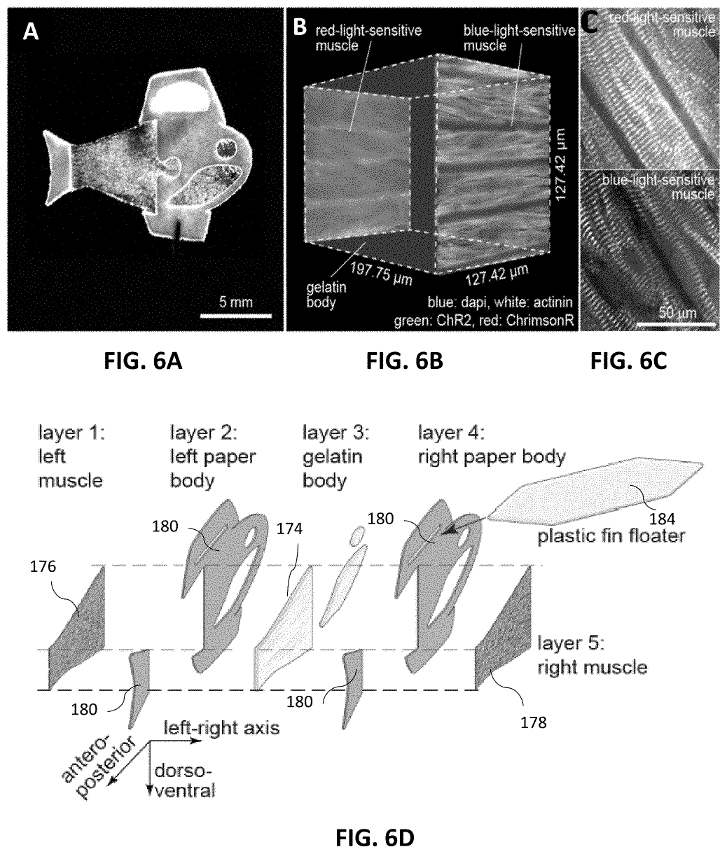

In some embodiments, a self-propelled swimming structure includes a multilayer engineered tissue structure that exhibits antagonistic cyclic contractions as described herein. FIG. 5 schematically depicts design of a self-propelled swimming structure in the form of a biohybrid fish incorporating a multilayer engineered tissue structure in accordance with some embodiments. FIG. 6D schematically depicts assembly of the biohybrid fish. The self-propelled swimming structure 170 includes a multilayer engineered cardiac tissue structure 172 including a support 174, a first engineered tissue layer 176, and a second engineered tissue layer 178 (see FIGS. 5F and 6D), a front body portion 180 coupled to or attached to a first end 172a of the engineered tissue structure, and a rear body portion 182 coupled to or attached to a second end 172b of the engineered tissue structure (see FIGS. 5F and 6D). In some embodiments, neither of the engineered tissue layers includes a G-node. In some embodiments, one of the engineered tissue layers includes a G-node. In some embodiments, each of the engineered tissue layers includes a G-node. In some embodiments, the engineered tissue layers include cardiomyocytes.

In some embodiments, a young's modulus of the front body portion is greater than a young's modulus of the support 110. In some embodiments, a young's modulus of the rear body portion is greater than the young's modulus of the support 110. In some embodiments, the self-propelled swimming structure further includes at least one buoyancy element 184 (e.g., a plastic floater fin of FIG. 6D) attached to or coupled to the front body portion, the rear body portion, or both. The at least one buoyancy element is selected for the self-propelled swimming structure to maintain neutral buoyancy in a culture medium. In some embodiments, the at least one buoyancy element is shaped to maintain directional stability of the self-propelled swimming structure.

### Methods of Forming a Functional Engineered Tissue Structure

Some embodiments provide a method of forming a functional engineered tissue structure (e.g., a functional cardiac tissue structure, a functional muscle tissue structure, a functional neural tissue structure). The method includes providing or obtaining a support having a first side and a second side opposite the first side, the first side being patterned and configured to promote anisotropic tissue formation, and the second side being patterned and configured to promote anisotropic tissue formation. Further description of patterning and configuring to promote anisotropic tissue function can be found in the section below entitled Seeding and Culturing of Engineered Tissue Layers and in the Example. The method further includes seeding the first side of the support and the second side of the support with excitable and contractile cells (e.g., cardiomyocytes, skeletal muscle cells), and growing a first tissue layer (e.g. a cardiac tissue layer, a skeletal muscle tissue layer) on the first side of the support and growing a second tissue layer (e.g. a cardiac tissue layer, a skeletal muscle tissue layer) on the second side of the support, the first cardiac tissue layer physically separated from the second cardiac tissue layer by a thickness of the support. Further description of seeding the cells and growing the tissue layers can be found in the section below entitled Seeding and Culturing of Engineered Tissue Layers and in the Example.

In some embodiments, during growth of the first tissue layer 176 on the first side of the support 174 and growth of the second tissue layer 178 on the second side of the support 174, excitation and contraction of the first tissue layer 176 bends the support 174 and strains the second tissue layer 178 inducing excitation and contraction of the second tissue layer 178, and the excitation and contraction of the second tissue layer 178 bends the support 174 and strains the first tissue layer 176 inducing excitation and contraction of the first tissue layer producing antagonistic cyclic contractions. In some embodiments, the antagonistic cyclic contractions are self-sustaining cyclic contractions. In some embodiments, the antagonistic cyclic contractions are spontaneous, self-sustaining cyclic contractions. In some embodiments, forces exerted by the self-sustaining cyclic contractions help to develop and sustain functional cell growth in the first tissue layer and the second tissue layer. Additional description of self-sustaining cyclic contractions sustaining functional cell development and growth over extended times is provided in the Example.

### Seeding and Culturing of Engineered Tissue Layers

The supports are seeded with a population of cells to fabricate the first engineered tissue layer and the second engineered tissue layer in engineered tissue structures in some embodiments.

Accordingly, in some embodiments, a support is seeded with a plurality of cells and cultured in an incubator under physiologic conditions (e.g., at 37° C.) until the cells form a functional tissue engineered tissue layer.

A functional tissue structure is an in vitro tissue that recapitulates one or more interactions that occur between cells and their surrounding tissue in vivo. For example, the sarcomeres in the muscle cells and/or the cell themselves of a functional muscle tissue layer may be anisotropically aligned and/or the tissue is electrically functional and actively contractile.

Any appropriate cell culture method may be used. The seeding density of the cells will vary depending on the cell size and cell type, but can easily be determined by methods known in the art. In one embodiment, cells are seeded at a density of between about 1×10⁵ to about 6×10⁵ cells/cm², or at a density of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶. Values and ranges intermediate to the above-recited values and ranges are also contemplated by the present invention.

In some embodiments, the support is contacted with living cells during the fabrication process such that engineered tissue layers populated with cells are produced. The support may also be contacted with additional agents, such as proteins, nucleotides, lipids, drugs, pharmaceutically active agents, biocidal and antimicrobial agents during the fabrication process.

Suitable cells for use in the invention may be normal cells, abnormal cells (e.g., those derived from a diseased tissue, or those that are physically or genetically altered to achieve an abnormal or pathological phenotype or function), normal or diseased muscle cells, stem cells (e.g., embryonic stem cells), or induced pluripotent stem cells. Suitable cells include vascular smooth muscle cells, cardiac myocytes, skeletal muscle cells, and cells that will differentiate into muscle cells. Such cells may be seeded on the support and cultured to form a functional tissue.

Cells for seeding can be cultured in vitro, derived from a natural source, genetically engineered, or produced by any other means. Any natural source of cells may be used.

The term “progenitor cell” is used herein to refer to cells that have a cellular phenotype that is more primitive (e.g., is at an earlier step along a developmental pathway or progression than is a fully differentiated cell) relative to a cell that it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “progenitor cell” is used herein synonymously with “stem cell.”

The term “stem cell” as used herein, refers to an undifferentiated cell that is capable of proliferation and giving rise to more progenitor cells having the ability to generate a large number of mother cells that can in turn give rise to differentiated, or differentiable daughter cells.

The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and that retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see U.S. Pat. Nos. 5,843,780, 6,200,806, the contents of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

Hydrogels suitable for use in the engineered tissue structures of the invention include, for example, polyacrylamide gels, poly(N-isopropylacrylamide), poly(hydroxyethyl) methacrylate (pHEMA), collagen, fibrin, gelatin, alginate, and dextran. In some embodiment the hydrogel includes gelatin. In some embodiment, the hydrogel includes alginate. In some embodiments, the stiffness of the hydrogel is tuned to mimic the mechanical properties of healthy muscle tissue, e.g., cardiac tissue in vivo, e.g., to have a Young's modulus of about 10, 11, 12, 13, 14, 15, 16, 17, 18, 19, or about 20 kPa. In some embodiments, the stiffness of the hydrogel is tuned to mimic the mechanical properties of diseased muscle tissue, e.g., cardiac tissue in vivo, e.g., to have a Young's modulus of greater than about 45, 46, 47, 48, 49, 50, 51, 52, 53, 54, or about 55 kPa.

A surface of a hydrogel layer may be uniformly or selectively patterned with engineered surface chemistry to elicit, e.g., an extracellular matrix protein, (or inhibit, e.g., a Pluronic) specific cell growth and function. The engineered surface chemistry can be provided via contact printing, exposure to ultraviolet radiation or ozone or via acid or base wash or plasma treatment to increase the hydrophilicity of the surface. In some embodiments, the hydrogel is micro-molded to have selective spatially patterned dimensions to elicit (or inhibit) specific cell growth and function. In some embodiments, a surface of the support is patterned to form anisotropic tissue.

A specific biopolymer (or combination of biopolymers) may be selected to recruit different integrins, or an engineered surface chemistry may be fabricated on the flexible polymer layer and/or hydrogel layer to enhance or inhibit cell and/or protein adhesion. The specific type of biopolymer used and geometric spacing of the patterning will vary with the application. In one embodiment, the engineered surface chemistry comprises a biopolymer, such as an ECM protein, to pattern specific cell types. The ECM may comprise fibronectin, laminin, one or more collagens, fibrin, fibrinogen, or combinations thereof.

In some embodiments, prior to uniformly or selectively patterning a hydrogel with an engineered surface chemistry to elicit, e.g., an extracellular matrix protein, (or inhibit, e.g., a Pluronic) specific cell growth and function, the hydrogel is coated with a compound to permit covalent attachment of an engineered surface chemistry, such as an extracellular matrix protein.

In one embodiment, the ECM is not uniformly distributed on the surface of the flexible polymer and/or hydrogel, but rather is patterned spatially using techniques including, but not limited to, soft lithography, self-assembly, printed on the solid support structure with a polydimethylsiloxane stamp, vapor deposition, and photolithography. Methods of forming an anisotropic tissue layer are describe in U.S. Patent Application Publication No. 2014/0342394, entitled “Muscle Chips and Methods of Use”, which is incorporated by reference herein in its entirety. Additional methods of forming anisotropic tissue layers on a support are described below with respect to the Example.

To attach cells, a support is placed in culture with a cell suspension allowing the cells to settle and adhere to the surface. In the case of an adhesive surface treatment, cells bind to the material in a manner dictated by the surface chemistry. For patterned chemistry, cells respond to patterning in terms of maturation, growth and function. Examples of cell types that may be used include contractile cells, such as, but not limited to, vascular smooth muscle cells, vascular endothelial cells, myocytes (e.g., cardiac myocytes), neurons, skeletal muscle, and cells that will differentiate into contractile cells (e.g., stem cells, e.g., embryonic stem cells or adult stem cells, progenitor cells or satellite cells).

In one embodiment, cardiac myocytes are co-cultured with neurons to prepare innervated engineered tissue comprising pacemaking cells, and/or to accelerate the maturation of the cultured cells as described in U.S. Patent Application Publication No. 20130046134-A1, filed Oct. 31, 2012, the entire contents of which are incorporated herein by reference.

Methods for Identifying a Compound that Modulates a Tissue Function and Methods for Identifying a Compound Useful for Treating or Preventing a Cardiac Tissue Disease

Some embodiments described herein include methods for identifying a compound that modulates a tissue function (e.g., a cardiac tissue function, a muscle tissue function, a neural tissue function). A method includes providing an engineered tissue structure as described herein and contacting the engineered tissue structure with a test compound. In some embodiments, the engineered tissue structure includes a first engineered tissue layer (e.g., a first engineered cardiac tissue layer, a first engineered muscle tissue layer, a first engineered neural tissue layer) on a first side of a support including a geometrically isolated node. In some embodiments, the engineered tissue structure is a multilayer tissue structure including a first engineered tissue layer (e.g., a first engineered cardiac tissue layer, a first engineered muscle tissue layer) on a first side of a support and second engineered tissue layer (e.g., a second engineered cardiac tissue layer, a second muscle tissue layer) on a second side of the support. The method also includes determining the effect of the test compound on the tissue function (e.g., cardiac tissue function, muscle tissue function) in the presence and absence of the test compound, wherein a modulation of the tissue function (e.g., cardiac tissue function, muscle tissue function) in the presence of the test compound as compared to the tissue function in the absence of the test compound indicates that the test compound modulates the cardiac tissue function, thereby identifying a compound that modulates a cardiac tissue function.

Some embodiments described herein include methods for identifying a compound useful for treating or preventing a tissue disease (e.g., a cardiac tissue disease, a muscular tissue disease, a neural tissue disease). A method for identifying a compound useful for treating or preventing a tissue disease includes providing an engineered tissue structure (e.g., an engineered cardiac tissue, an engineered muscle tissue, an engineered neural tissue), and contacting the engineered tissue structure with a test compound. In some embodiments, the engineered tissue structure includes a first engineered tissue layer (e.g., a first engineered cardiac tissue layer, a first engineered muscle tissue layer, a first engineered neural tissue layer) on a first side of a support including a geometrically isolated node. In some embodiments, the engineered tissue structure is a multilayer tissue structure including a first engineered tissue layer (e.g., a first engineered cardiac tissue layer, a first engineered muscle tissue layer) on a first side of a support and second engineered tissue layer (e.g., a second engineered cardiac tissue layer, a second muscle tissue layer) on a second side of the support. The method also includes determining the effect of the test compound on a tissue function (e.g., a cardiac tissue function, a muscle tissue function, a neural tissue function) in the presence and absence of the test compound, where a modulation of the tissue function in the presence of the test compound as compared to the tissue function in the absence of the test compound indicates that the test compound modulates the tissue function, thereby identifying a compound useful for treating or preventing a tissue disease.

In some embodiments in which the cells are contractile cells, a deflection, a movement, or a change in curvature of the support due to contraction of the first engineering tissue layer and the second engineering tissue layer may be used to measure the tissue function. In some embodiments in which the cells are contractile cells, a magnitude of a deflection, a movement, or a change in curvature of the support may be used to measure the tissue function. In some embodiments in which the cells are contractile cells, a frequency of deflections, movements, or changes in curvature of the support may be used to measure the tissue function. In some embodiments, both a magnitude of a frequency of deflections, movements, or changes in curvature of the support may be used to measure the tissue function. Additional description of measurements of tissue function are described in U.S. Patent Application Publication No. 2014/0342394, entitled “Muscle Chips and Methods of Use”, which is incorporated by reference herein in its entirety. Additional description of measurements of tissue function are provided in the Example below.

In some embodiments, the tissue function may be a biomechanical activity. The biomechanical activity may be one or more of contractility, cell stress, cell swelling, and rigidity. The biomechanical activity may also be one or more of stem cell activation, stem cell maturation, tissue morphogenesis, and tissue remodeling. In other embodiments, the tissue function may be an electrophysiological activity. The electrophysiological activity may be a voltage parameter selected from the group including action potential, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a scaffold including pancreatic islet cells or adipocytes with a test compound) is intended to include any form of interaction (e.g., direct or indirect interaction) of a test compound and a scaffold or cells. The term contacting includes incubating a compound and scaffold or tissue together (e.g., adding the test compound to scaffold including pancreatic islet cells or adipocytes in culture).

Test compounds can be any agents including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The engineered tissue structure and/or the population(s) of cells can be contacted with a test compound by any suitable means. For example, the test compound can be added drop-wise onto the surface structure and allowed to diffuse into or otherwise enter the structure, or it can be added to the nutrient medium and allowed to diffuse through the medium.

This invention is further illustrated by the following Example, which should not be construed as limiting.

### Example. An Autonomously Swimming Biohybrid Fish Designed with Human Cardiac Biophysics

The circulatory system of organisms is intricately designed to transport blood throughout the body. Its most basic function is fluid transport, and a diversity of similar fluid pumping mechanisms and designs are found throughout nature. Fluid pumps in vertebrates, considered broadly, range from a human circulatory system with closed vessels within which fluid moves, to oscillatory fluid mechanisms in aquatic species in which fluid is transported along the body to generate propulsive thrust. Inspired by these distinct but similar natural processes, biohybrid analogs of an external fluid pump capable of mimicking the locomotion of aquatic species have been developed (J. C. Nawroth et al., Nat Biotechnol 30, 792-797 (2012); S. J. Park et al., Science (New York, N.Y.) 353, 158-162 (2016); L. Ricotti et al., Science Robotics 2, eaaq0495 (2017)). The underlying motivation for developing biohybrid systems capable of reproducing biological behaviors has been to better understand the design principles and coordination mechanisms of biological systems, although their performance has been lacking in comparison to natural fluid transport pumps (L. Ricotti et al., Science Robotics 2, eaaq0495 (2017)).

A key feature of aquatic species is closed-loop actuation of antagonistic musculature that provides control over the direction of momentum transfer from the body muscles to the fluid enabling efficient locomotion. Similarly, in the circulatory system, muscles of the heart dynamically respond to physiological demands through internal feedback systems and impart momentum to drive fluid motion. Mechano-electrical signaling and cardiac automaticity play an essential role in regulating the contractile pace and strength in a closed-loop control system (FIG. 5A). Mechano-electrical signaling (H. E. ter Keurs, Pflugers Arch 462, 165-175 (2011); T. A. Quinn et al., Physiol Rev 101, 37-92 (2021)) is hypothesized to regulate intra-cardiac feedback, which allows cardiomyocytes (CMs) to adaptively respond to dynamic mechanical pressures (T. A. Quinn et al., Prog Biophys Mol Biol 115, 71-75 (2014); B. E. Dabiri et al., Prog Biophys Mol Biol 110, 196-203 (2012)) by inducing changes in electrophysiology via stretch-activated mechanosensitive proteins (O. Friedrich et al., Prog Biophys Mol Biol 110, 226-238 (2012); T. A. Quinn et al., Circulation. Arrhythmia and electrophysiology 10, (2017)) Automaticity of the heart stems from the sinoatrial node. The sinoatrial node, structurally and functionally insulated from the surrounding myocardium initiates spontaneous electrical activity in the absence of an external stimulus and without direct neural intervention/

Using principles of cardiac control systems to design a biohybrid platform could result in a fluid pumping system with comparable efficiencies to natural fish-like fluid pumping systems. Leveraging fundamental features of cardiac function allows for autonomous self-pacing and independent motion control, while providing the basis for a closed-loop design that mimics aquatic swimming systems. A biohybrid fish (FIG. 5D) equipped with an antagonistic muscular bilayer (FIG. 5B) and a geometrically insulated cardiac tissue node (G-node) (FIG. 5C) using human stem cell-derived CMs or neonatal rat ventricular CMs was designed, built, and tested to test the ability of a biohybrid system to move fluid with control and biological levels of performance. To integrate mechano-electrical signaling of CMs in a simplified biohybrid platform, asynchronous muscle contractions (FIG. 5C) were recreated, inspired from insects' muscle movements (B. Bullard et al., J. Muscle Res. Cell Motil. 32, 303-313 (2011)). In insects, each contraction results automatically from a response to the stretching of an antagonistic muscle pair, generating self-sustained muscle contraction cycles. In the muscular bilayer construct of the biohybrid fish (FIG. 5B), CMs are electrically connected within each side and mechanically coupled across sides, so that the shortening of contracting muscles on each side directly translates to axial stretching of the opposite side muscle, leading to antagonistic muscle excitations and contractions. To replicate the electrically insulated structure of a sinoatrial node (FIG. 5A) (R. W. Joyner et al., Biophysical journal 50, 1157-1164 (1986)), a small number of CMs (the source) were functionally isolated in the G-node (FIG. 5C) with a single exit pathway that allows for an electrical connection between the G-node and muscle tissue (the sink). This facilitated the activation of large downstream quiescent muscle cells (sink) with a small number of activating CMs (source) by reducing the impedance between source and sink (S. D. Unudurthi et al., Frontiers in physiology 5, 446 (2014); A. G. Kléber et al., Physiol Rev 84, 431-488 (2004); S. Rohr et al., Science (New York, N.Y.) 275, 841-844 (1997); R. W. Joyner et al., Biophysical journal 50, 1157-1164 (1986); materials and methods are described below). Together, the muscular bilayer and G-node in the biohybrid fish (FIG. 5D) enabled the generation of continuous rhythms to regulate its antagonistic muscle pair to produce spontaneous yet, coordinated, body-caudal fin (BCF) propulsion swimming.

### A. Antagonistic Contraction of Muscular Bilayer Construct

A muscular bilayer construct was developed by modifying hydrogel-based muscular thin films (S. J. Park et al., Circulation 140, 390-404 (2019); M. L. McCain et al., Biomaterials 35, 5462-5471 (2014); materials and methods are described below). The double-sided micromolded gelatin thin film (200 μm thick) was engineered by sandwiching a gelatin and crosslinker (microbial transglutaminase) mixture between two polydimethylsiloxane (PDMS) stamps with line groove features (25 μm ridge width, 4 μm groove width, and 5 μm groove depth). Then, CMs were seeded onto both sides of the micromolded gelatin so that CMs could self-assemble as laminar, anisotropic muscle with engineered cellular alignment, characteristic of the ventricular myocardium (FIGS. 6A and 6B).

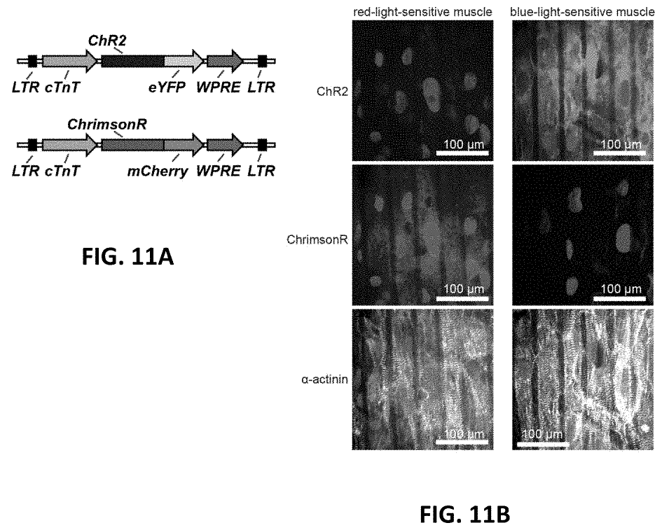

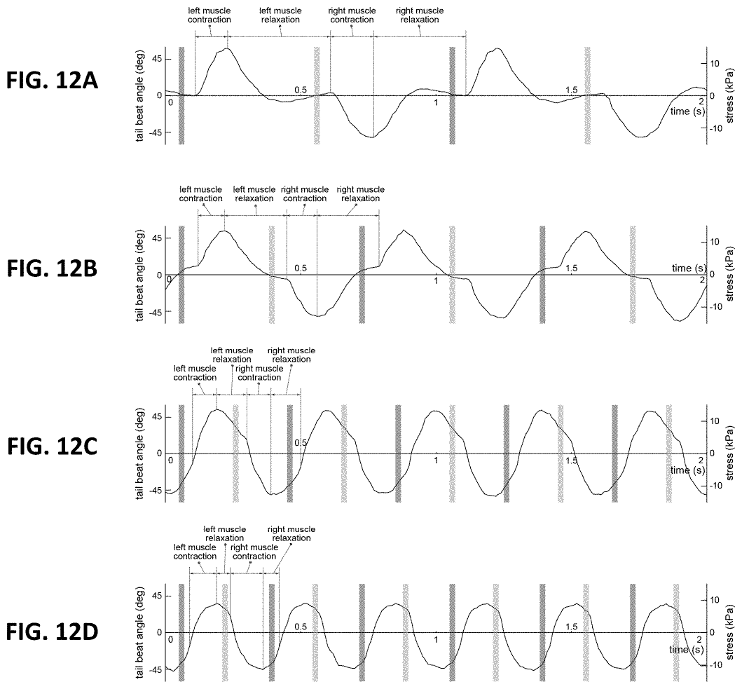

To demonstrate independent activation between the muscular bilayer tissues, blue-light-sensitive (ChR2 (E. S. Boyden et al., Nat Neurosci 8, 1263-1268 (2005)) and red-light-sensitive (ChrimsonR (N. C. Klapoetke et al., Nature methods 11, 338-346 (2014)) ion channels were expressed in each muscle layer using lentiviral transduction (FIGS. 6 B and 6C, FIG. 11). Alternating blue and red light stimulation (15-msec pulses of 450 nm and 620 nm light) activated ChR2- and ChrimsonR-expressing muscle layers independently. The shortening of contracting muscles on each side was transduced to produce antagonistic bending stress and oscillate the muscle construct along the longitudinal axis (FIG. 12). The contractions and relaxations of muscular bilayer muscles were decoupled at low pacing frequencies (e.g., 1 and 1.5 Hz), but at higher pacing frequencies (e.g., 2.5 and 3 Hz), the relaxation of one-side started to overlap with the subsequent contraction of the other-side (FIG. 12). The overlapping fast active contraction of the opposite side muscle dramatically increased the oscillating speed of the muscular bilayer construct (FIG. 12), preventing diastolic stress development that single layered muscular thin films exhibit at high pacing frequencies (S. J. Park et al., Circulation 140, 390-404 (2019); M. L. McCain et al., Biomaterials 35, 5462-5471 (2014)). These antagonistic muscle contractions in muscular bilayer construct permitted large peak to peak amplitudes over a wide range of pacing frequencies (FIG. 12), in contrast to single layered muscular thin films (S. J. Park et al., Circulation 140, 390-404 (2019); M. L. McCain et al., Biomaterials 35, 5462-5471 (2014)).

B. Integration of Muscular Bilayer into Biohybrid Fish

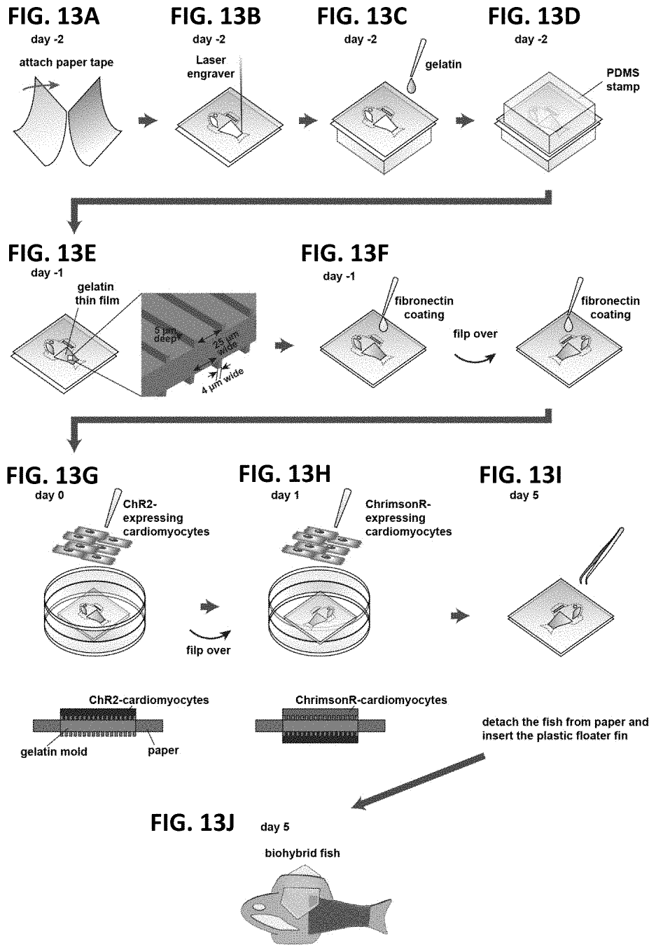

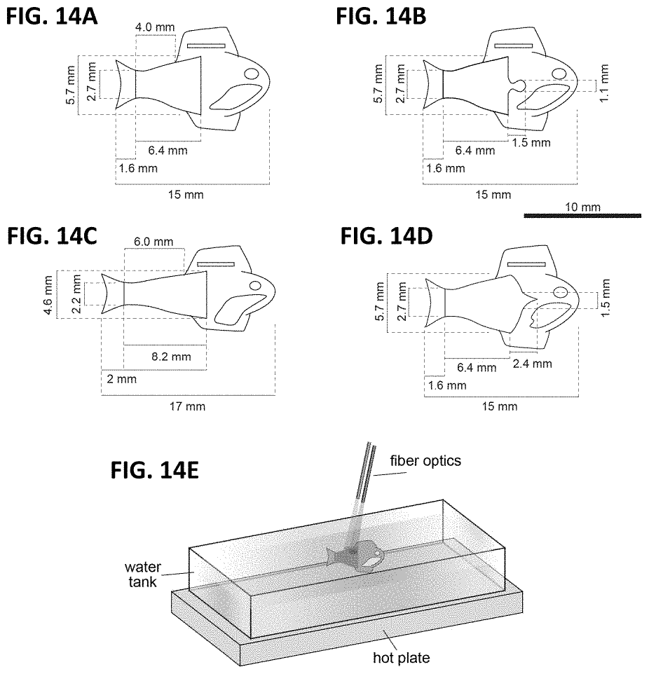

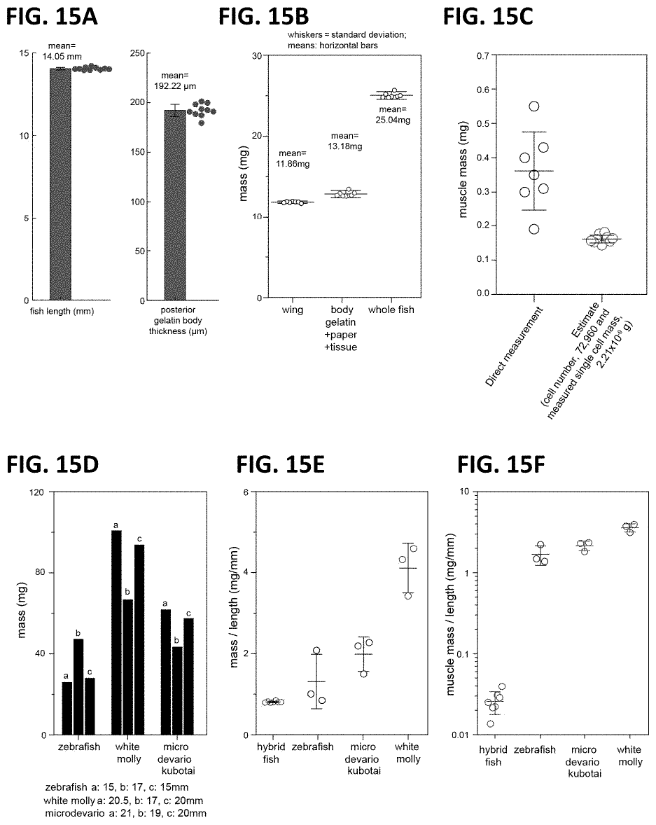

The muscular bilayer construct was integrated into the biohybrid fish (as descried below) using tissue engineering techniques (FIG. 13). Inspired by a fish's musculoskeletal structure, an asymmetrical body was created along both the antero-posterior and dorso-ventral axis, while maintaining sagittal symmetry via a five-layered architecture. From left to right (FIG. 6D), the biohybrid fish consists of (1) a layer of aligned muscle tissue made of human stem cell-derived CMs; (2) a rigid paper layer in the anterior body and caudal fin fabricated by laser ablation; (3) a compliant gelatin layer in the posterior body cast via a three-dimensional elastomer polydimethylsiloxane mold; (4) a second paper layer; (5) a second aligned muscle tissue layer for forming the antagonistic muscle pair. The passive component of the biohybrid fish comprising paper (thickness: 190 μm, Young's modulus: 4 GPa, density: 1.2 g/ml), gelatin body (thickness: 192.22±1.95 μm, Young's modulus: 56 kPa, density: 1.5 g/ml), and a plastic floater fin (thickness: 1 mm, Young's modulus: 1.3 GPa, density: 0.833 g/ml) were designed to maintain directional body stability and neutral buoyancy while minimizing drag during forward swimming. The large surface area of the floater fin combined with the relatively heavy weight of the hydrogel insert in the anterior ventral portion of the body helped the fish maintain an upright orientation. Neutral buoyancy was achieved by adjusting the size of the plastic floater fin, thereby matching the average density of the biohybrid fish to the media in which it was suspended. The active component of the biohybrid fish consists of muscular bilayer construct on the flexible posterior gelatin hydrogel body and operates as a single self-propelling system through coordinated contraction of muscle tissues. The final overall design (FIG. 14A-14E) consists of 73,000 live CMs in a hydrogel-paper composite body of 14 mm length and 25.0 mg total mass including 0.36 mg muscle mass (FIG. 15A-15F).

### C. Optogenetically Induced BCF Propulsion

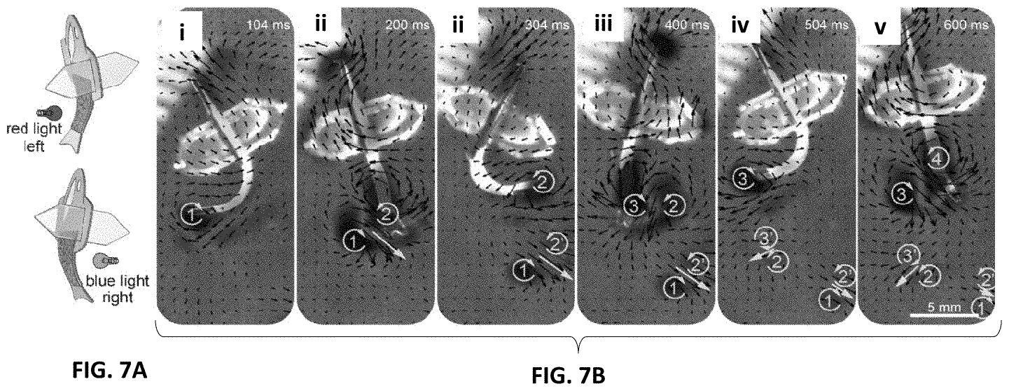

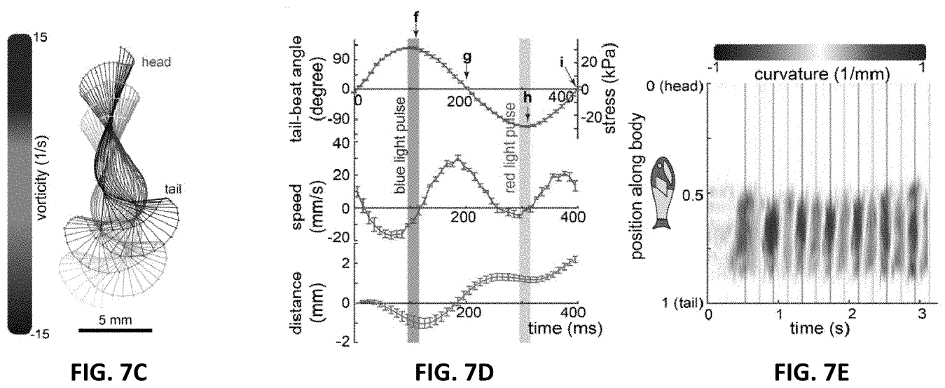

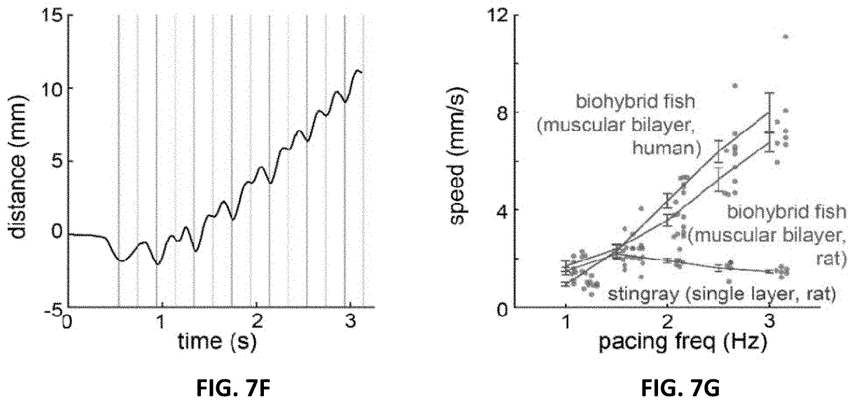

To characterize the system-level kinematics of the muscular bilayer, antagonistic muscle contractions in the biohybrid fish was controlled by external optogenetic stimulation (FIG. 7A-7L). The muscular bilayers were stimulated by alternating blue and red LED light pulses (FIG. 7A), while submerged in a 37° C. Tyrode's salt solution containing glucose. As shown in the video-tracking analysis (FIGS. 7B-7H), the biohybrid fish (1) initiated contraction of the left-sided muscle tissue upon red light stimulation and produced a peak oscillation amplitude in the tail (FIGS. 7B, 7F, and 7I); (2) the blue light stimulation induced contraction of the right-sided muscle tissue (180° phase shift between red and blue lights); (3) recovered its tail at a near-straight position (FIGS. 7C and 7G) reaching peak thrust production (FIG. 7I); (4) oscillated its tail with peak amplitude right before a subsequent red light stimulation (FIGS. 7D and 7I); and (5) rebounded back to a near-straight position (FIG. 7E) generating maximal thrust (FIG. 7I). As shown by the lateral deflection (FIG. 7H), the body curvature (FIG. 7J), and swimming displacement (FIG. 7K), the biohybrid fish generated a rhythmic forward thrust by reproducing BCF propulsion. The biohybrid fish deformed its posterior body with a single-bend while switching between positive and negative posterior body curvature upon light stimulation. The biohybrid fish oscillated its fin instead of generating a bending body wave because optical stimulation induced a simultaneous global muscle contraction. The relatively stiff anterior body and caudal fin resisted deformation due to fluid forces. This allowed the biohybrid fish to exhibit asymmetric body deformation in which the largest lateral deflections and curvatures occurred in the posterior body between 0.5 and 0.8 of total length (FIG. 7J), in a manner reminiscent of BCF swimmers.

Antagonistic muscle contractions of the biohybrid fish generated a hydrodynamic signature like those of wild-type BCF swimmers—specifically the water flow in the wake and around the fish bodies which were visualized using particle image velocimetry (PIV) (FIGS. 7B-7H). The biohybrid fish shed two vortex pairs per tail-beat cycle and one pair per lateral tail excursion, one of the key characteristic flow patterns of BCF swimmers. Each lateral tail excursion from bent to near-straight positions induced strong wake flows that formed a visible vortex pair with the opposite rotational direction (FIGS. 7B and 7C, vortices 1 and 2′; FIGS. 7 D and 7E, vortices 2 and 3′; FIGS. 7F and 7G, vortices 3 and 4). When the vortex pair reached the tail from the posterior body, it was shed (FIG. 7D vortices 1 and 2′; FIG. 7F, vortices 2 and 3′) and continuously moved away from the fish body under its own momentum (FIGS. 7D-7G vortices 1 and 2′; FIGS. 7F and 7G, vortices 2 and 3′).

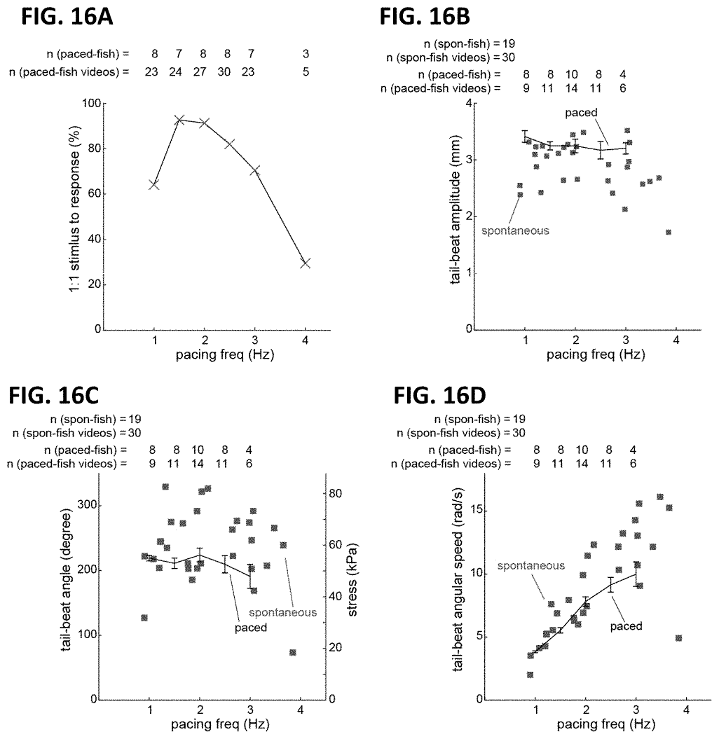

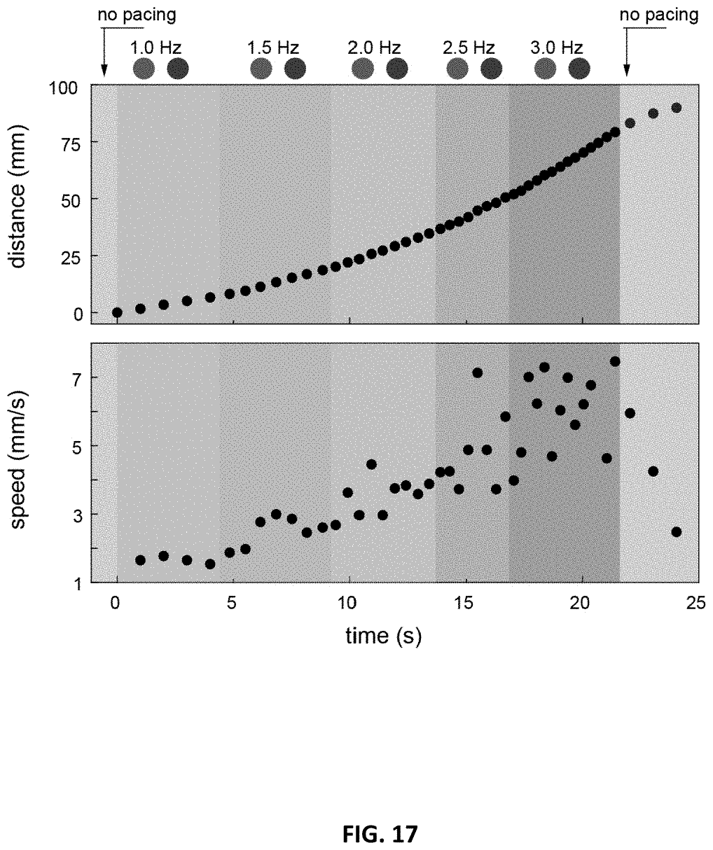

The inclusion of a muscular bilayer architecture improved the high-frequency swimming of the biohybrid fish. The biohybrid fish (6.4 mm long muscle tissue body) was optogenetically controlled responded up to 3-4 Hz (FIG. 16), maintained a large tail-beat amplitude and angle, and exhibited a positive pacing frequency and tail-beat angular speed relationship (FIG. 16). The biohybrid fish made of human stem cell-derived CMs and primary neonatal rat ventricular CMs (FIG. 17) exhibited increased swimming speeds with increasing pacing frequencies (FIG. 7L) reminiscent of the force-frequency relationship of the heart. In contrast, a previous biohybrid stingray (S. J. Park et al., Science (New York, N.Y.) 353, 158-162 (2016)) exhibited reduced swimming speeds at high pacing frequencies due to having a single-layered muscle, lacking antagonistic muscle contractions. The upper limit of optogenetic pacing frequency that induces a 1:1 stimulus response is also affected by the body dimensions: a longer body fish (8.2 mm long, FIG. 14C) exhibited oscillatory motion up to 2 Hz, but not at 2.5 and 3 Hz stimulations.

### D. Autonomous BCF Propulsion

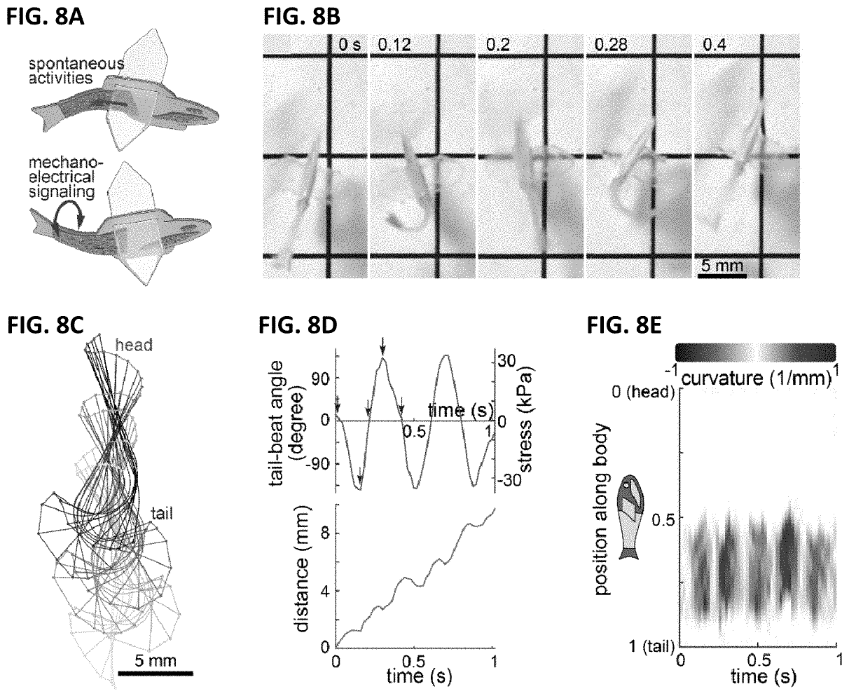

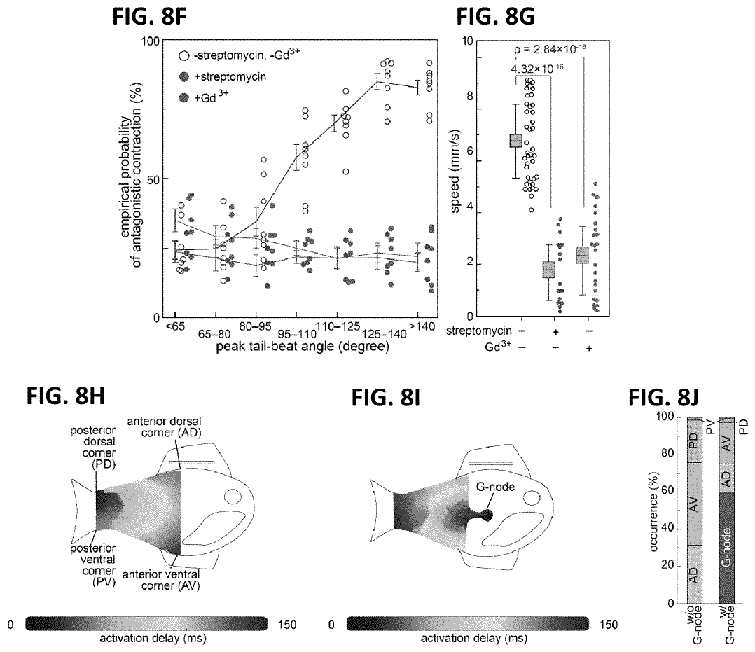

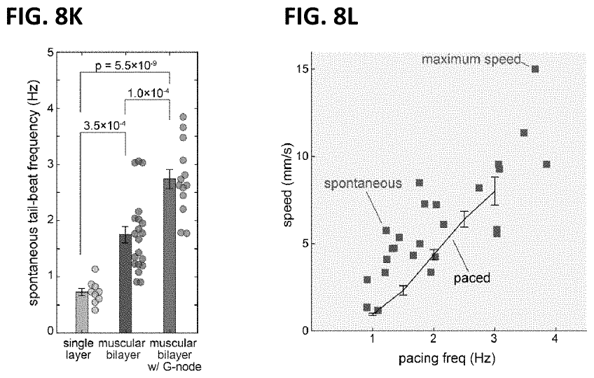

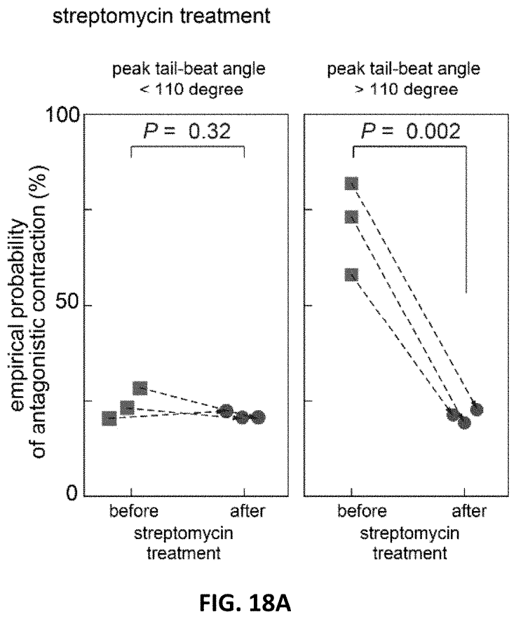

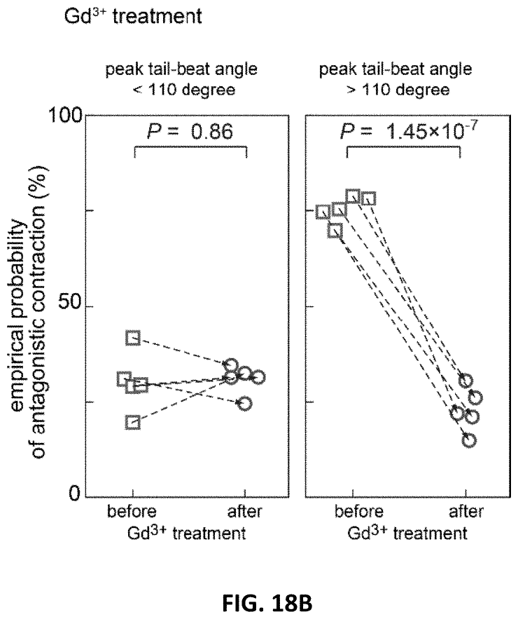

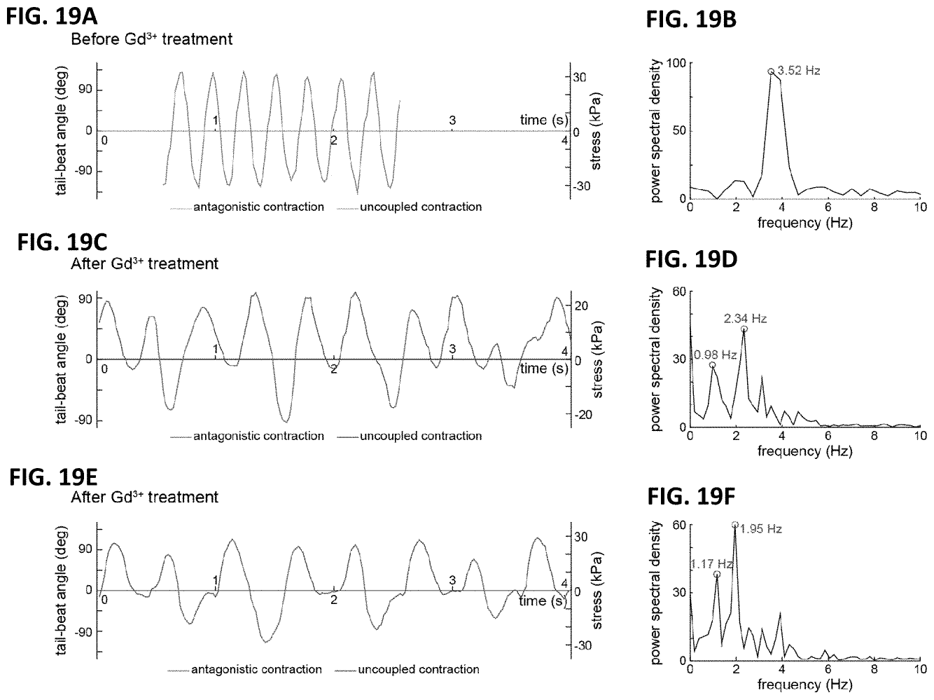

Whether reconstructing antagonistic muscle contractions with CMs could sustain spontaneous rhythmic contractions via mechano-electrical signaling was tested (FIG. 8A). Spontaneous activation and contraction on one-side of the 49-days old biohybrid fish led to a subsequent antagonistic contraction on the opposite side via mechanical coupling between muscle tissues (FIG. 8B). These spontaneous antagonistic contractions led to alternating bending motions of the posterior body (FIGS. 8C-8E) resulting in rhythmically sustained forward displacement (FIG. 8D) as shown in optogenetically triggered body-caudal fin propulsions (FIG. 7). Importantly, biohybrid fish with a larger tail-beat angle had a higher probability to induce a subsequent muscle contraction (FIG. 8F), suggesting that the lengthening of one muscle layer caused by a shortening of the other muscle layer directly induced subsequent contractions through cardiac mechano-electrical signaling. The biohybrid fish with stretch-activated ion channels inhibitors (streptomycin (F. Gannier et al., Cardiovascular research 28, 1193-1198 (1994)) and gadolinium (X. C. Yang et al., Science (New York, N.Y.) 243, 1068-1071 (1989)), Gd³⁺) was tested. It was observed that these inhibitors disrupted antagonistic contractions in the biohybrid fish by breaking the positive relationship between peak-tail-beat angle and probability of antagonistic contractions (FIG. 8F, FIG. 18). Further, frequency-domain analysis showed that the spontaneous frequencies of streptomycin and Gd³⁺-treated muscular bilayer tissues were not harmonic (FIG. 19). Stretched-activated ion channel inhibition decreased swimming speeds (FIG. 8G), which demonstrated that mechano-electrical signaling mediates self-sustainable spontaneous rhythmic contractions in muscular bilayers.

Whether reconstructing a geometrically distinct and electrically insulated node could initiate spontaneous electrical activity due to the automaticity of CMs in the absence of an external stimulus was tested. Inspired by the partial electrical insulation of a sinoatrial node (R. W. Joyner et al., Biophysical journal 50, 1157-1164 (1986)), the G-node (FIG. 5E) was created, where a small number of CMs are structurally and functionally isolated with a single exit pathway. The G-node is electrically coupled by gap junctions (A. G. Kléber et al., Physiol Rev 84, 431-488 (2004); A. G. Kléber et al., Biophysics Reviews 2, 031301 (2021)) to muscle tissues and facilitates progressive activation of large quiescent neighboring muscle cells (sink) by a small number of activating CMs (source). The geometrical design of both the G-node and the sink is crucial in determining the leading muscle activation site, because the electrical current being exchanged between individual CMs of different membrane potentials can be reflected at the tissue edges (A. G. Kléber et al., Physiol Rev 84, 431-488 (2004); materials and methods described below). Thus, the reflection of intracellular currents at the perimeter of G-node would synchronize the spontaneous activity and initiate coordinated pacemaking from the G-node.

To decouple the effect of antagonistic muscle contractions to muscle activation from the G-node, muscle movement with laboratory tape on a glass slide was mechanically restricted and muscle activation was determined using calcium imaging (Code and scripts. Zenodo (2021); http://doi.org/10.5281/zenodo.5618323/). CMs in the G-node and four corners (Anterior ventral corner: AV, Anterior dorsal corner: AD, Posterior ventral corner: PV, and Posterior dorsal corner: PD and FIGS. 20-22) of the muscle tissue overcame source-sink mismatch and initiated muscle activation (FIGS. 8H and I, FIGS. 20-22). The G-node predominantly activated the muscle construct over the other four corners of the muscle tissue (FIGS. 8I and 8J and FIGS. 20-22). Comparing two G-node sizes showed that a larger G-node containing about 1,700 cells, increased the probability of initial muscular activation at the G-node compared to the smaller G-node with a pointed node (˜600 cells) (FIG. 20) suggesting that a group of geometrically distinct CMs are needed to initiate muscular activation. Additionally, rounding the sink's corners decreased the probability of activation at the corners (FIG. 21) by increasing the number of downstream cells at each respective corner (FIG. 23), but the G-node's corner design did not affect probability of activation at the G-node (FIG. 22), which indicates that acute angles in the small source tissue like the G-node is not critical in determining the activation site. Rather, this suggests that the larger perimeter to area ratio of the G-node synchronized electrical interaction between the geometrically distinct CMs through reflections of electronic currents and produced a relatively fast and synchronized activation over the sink tissue. Acute angled anterior corners of the fish body increased the probability of activation at the anterior side (FIG. 8J) by decreasing the number of downstream cells (FIG. 23), thus allowing cells in the anterior side (G-node and anterior corners, AD and AV) to predominantly initiate spontaneous activation waves (60% from G-node and 97% from all anterior sides, FIG. 8J).

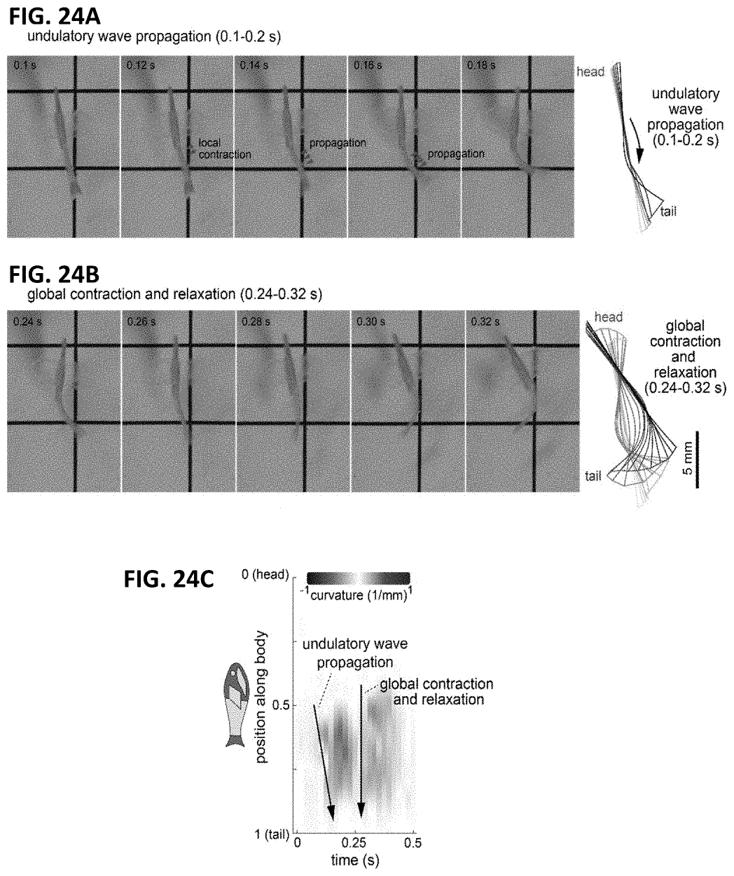

However, upon removing the restrictions on muscle movement, the G-node primarily acted as a secondary mechanism of controlling contractions. Only when the antagonistic muscle contractions were minimal, would the G-node initiate sequential local muscle activation and contraction, leading to undulatory locomotion (FIG. 24A). However, in subsequent muscle contractions, the biohybrid fish predominantly exhibited simultaneous global contractions and oscillatory locomotion with minimal body wave propagations, caused by mechano-electrical signaling of the muscular bilayer (FIG. 8E, FIGS. 24B and 24C). Although G-nodes are located on both sides of the tissues, one dominant G-node controlled initiation of muscle contraction as a secondary pacemaker. Due to the G-node's role as a secondary pacing mechanism of antagonistic contractions, biohybrid fish equipped with a G-node had significantly increased spontaneous contractile frequencies (FIG. 8K) while maintaining similar body kinematics (FIG. 24) and a positive frequency-swimming speed relationship like those of externally stimulated fish (FIG. 8L). As a result, the G-node-equipped biohybrid fish demonstrated increased maximum swimming speeds to more than one body length per sec (e.g., 15 mm/sec).

Although these G-node-entrained, mechano-electrical signaling-sustained, cyclic antagonistic muscle contractions are autonomous, optogenetic stimulation can be used for on-demand locomotion control. Antagonistic muscle contractions became coupled with optical pacing within less than three sequential light pulses. Further, optogenetic stimulation can also be used to inhibit autonomous locomotion—pausing right after a pulsed stimulation can stop muscle contractions for an extended period (e.g., 50 secs). Prolonged continuous optogenetic stimulation stops muscle contractions and autonomous locomotion. External stimulation reinitiates autonomous, antagonistic muscle contractions via activating mechano-electrical signaling.

### E. Advanced Performance of the Biohybrid Fish

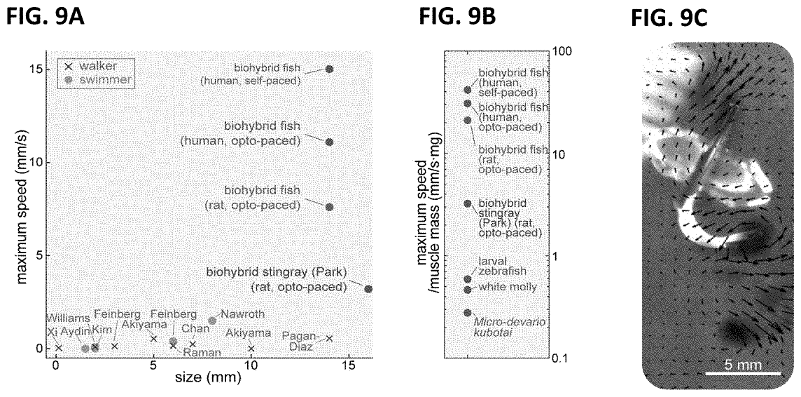

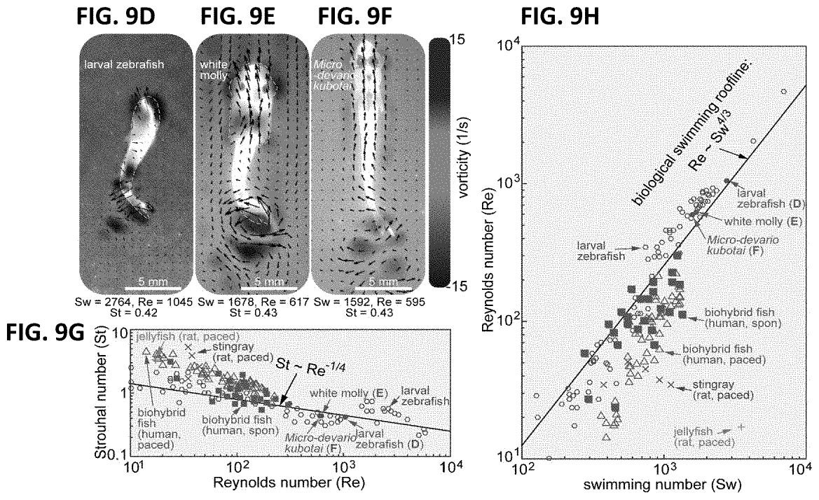

The autonomously swimming biohybrid fish (e.g., swimming at 15.0 mm/s) outperformed the locomotory speed of prior biohybrid muscular systems (J. C. Nawroth et al., Nat Biotechnol 30, 792-797 (2012); S. J. Park et al., Science (New York, N.Y.) 353, 158-162 (2016); A. W. Feinberg et al., Science (New York, N.Y.) 317, 1366-1370 (2007); J. Xi, J. J. Schmidt et al., Nature materials 4, 180-184 (2005); C. Cvetkovic et al., Proceedings of the National Academy of Sciences of the United States of America 111, 10125-10130 (2014); V. Chan et al., Scientific reports 2, 857 (2012); J. Kim et al., Lab on a chip 7, 1504-1508 (2007); Y. Akiyama et al., PloS one 7, e38274 (2012); Y. Akiyama et al., Biomed Microdevices 14, 979-986 (2012); G. J. Pagan-Diaz et al., Adv. Funct. Mater. 28, 13 (2018); R. Raman et al., Proceedings of the National Academy of Sciences of the United States of America 113, 3497-3502 (2016); B. J. Williams et al., Nature communications 5, 3081 (2014); O. Aydin et al., Proceedings of the National Academy of Sciences of the United States of America 116, 19841-19847 (2019)) (5-27× the speed of the biohybrid stingray (S. J. Park et al., Science (New York, N.Y.) 353, 158-162 (2016)) and the biohybrid skeletal muscle biorobot (G. J. Pagan-Diaz et al., Adv. Funct. Mater. 28, 13 (2018)) (FIG. 9A), highlighting the role of feedback mechanisms in developing biohybrid systems. Moreover, when considering the ratio of muscle mass to the total weight, biohybrid fish (1.4%, FIG. 15) and biohybrid stingray (9.7% ((S. J. Park et al., Science (New York, N.Y.) 353, 158-162 (2016))), the biohybrid fish demonstrated greater swimming speeds per unit muscle mass by an order of magnitude (13× over the maximum swimming speed of biohybrid stingray ((S. J. Park et al., Science (New York, N.Y.) 353, 158-162 (2016))) (FIG. 9B).

The biohybrid fish resembles the swimming performances of wild-type BCF swimmers with a similar body length (juvenile zebrafish, juvenile white molly, and Micro-devario kubotai) (FIGS. 9C-9F). Like the biohybrid fish, each of these species moves by shedding a pair of reverse-sign vortices when their tails reach maximum lateral excursion (FIGS. 9C-9F). The strength of these vortices between the biohybrid and wild-type fish were comparable (FIGS. 9C-9F). Rather than forming a continuous chaotic chain of the wakes, both the biohybrid and wild-type fish maintain stable vortex pairs with minimal vortex interactions (FIGS. 9C-9F). The stable wake pattern is a typical characteristic of juvenile zebrafish locomotion swimming at relatively high Strouhal numbers (St) and relatively low Reynolds numbers (Re<5000) (G. K. Taylor et al., Nature 425, 707-711 (2003)), where viscous forces cannot be neglected and the lateral velocity of wake flows are relatively high. In this flow regime, the swimming speed is nearly proportional to the tail-beat frequency (M. Gazzola et al., Nat. Phys. 10, 758-761 (2014)). Thus, the juvenile zebrafish, white molly, and Micro-devario kubotai with a faster tail-beat frequency (16.7 Hz, 7.5 Hz, and 7.7 Hz, which is 4.6×, 2.1×, and 2.1× over the biohybrid fish) showed proportionally increased swimming speeds of 59.7, 25.1, and 21.3 mm/s, respectively (4.0×, 1.7×, and 1.5× over the biohybrid fish). Although muscle functions in wild-type fish are beyond locomotion, when considering the ratio of total muscle mass to the total weight of biohybrid fish (1.4%, FIG. 15) compared with wild-type fish (80% (H. E. Jackson et al., Mech Dev 130, 447-457 (2013))), the maximum swimming speed per unit muscle mass of biohybrid fish exceeded those of wild-type fish by 70-150× (FIG. 9B).

### F. Efficiency of the Biohybrid Fish

To analyze the efficiency of the biohybrid fish, scaling and dimensional analysis was used. Wild-type swimmers achieved energetically favorable locomotion via convergent evolution and are found to hew to the two scaling relationships, St˜Re^{-1/4} and Re˜Sw^{-1/4} in the low Re and high St flow regime (M. Gazzola et al., Nat. Phys. 10, 758-761 (2014)) (FIGS. 9G and 9H). The Strouhal number St=fA/U (f is the tail-beat frequency, A is the tail-beat amplitude, and U is the forward speed) represents the ratio of the lateral oscillation amplitude to swimming distance per lateral tail excursion, the Swimming number Sw=2πfAL/υ (L is the characteristic body length of the swimmer, and υ is the fluid viscosity) represents input kinematics, and the Reynolds number Re=UL/υ compares inertial to viscous forces and is a function of swimming speed. Compared with the biohybrid stingray (S. J. Park et al., Science (New York, N.Y.) 353, 158-162 (2016)), the proposed biohybrid fish operates much closer to these average scaling relationships of wild-type swimmers. Moreover, the biohybrid fish swimming at high tail-beat frequencies (high St and Sw) reached a performance comparable to those of wild-type swimmers (FIGS. 9G and 9H).

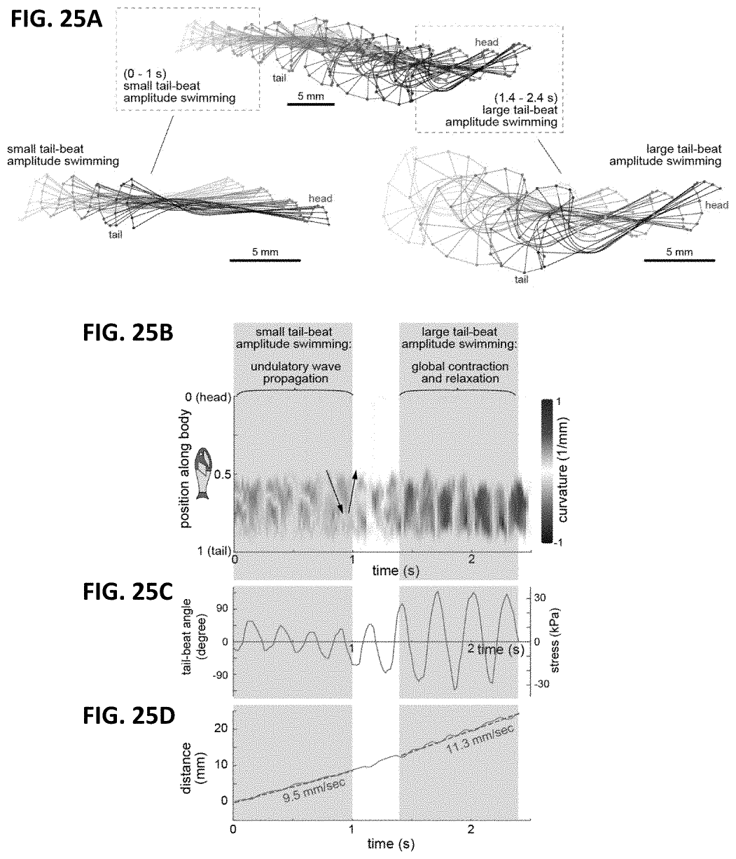

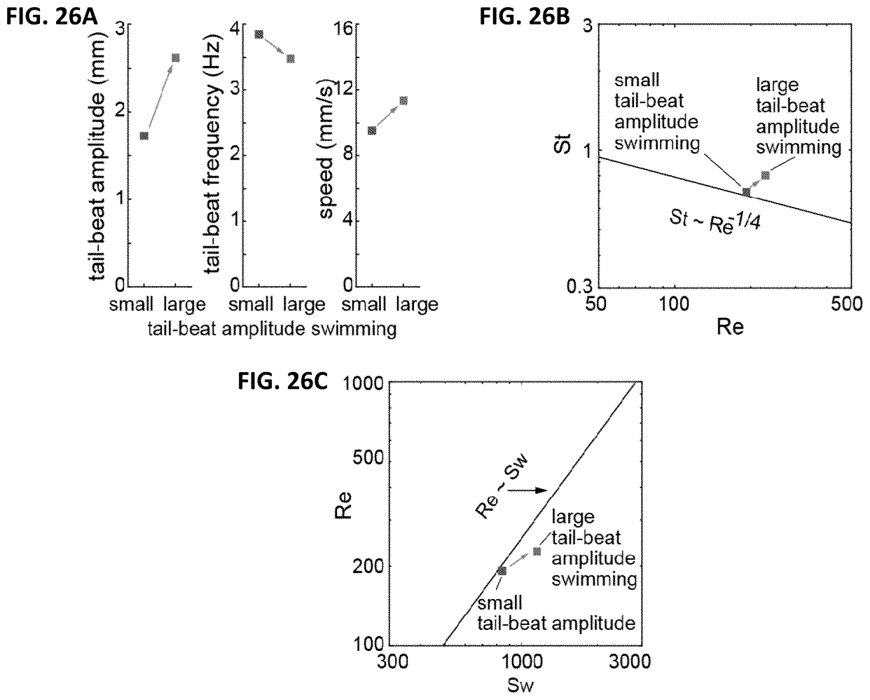

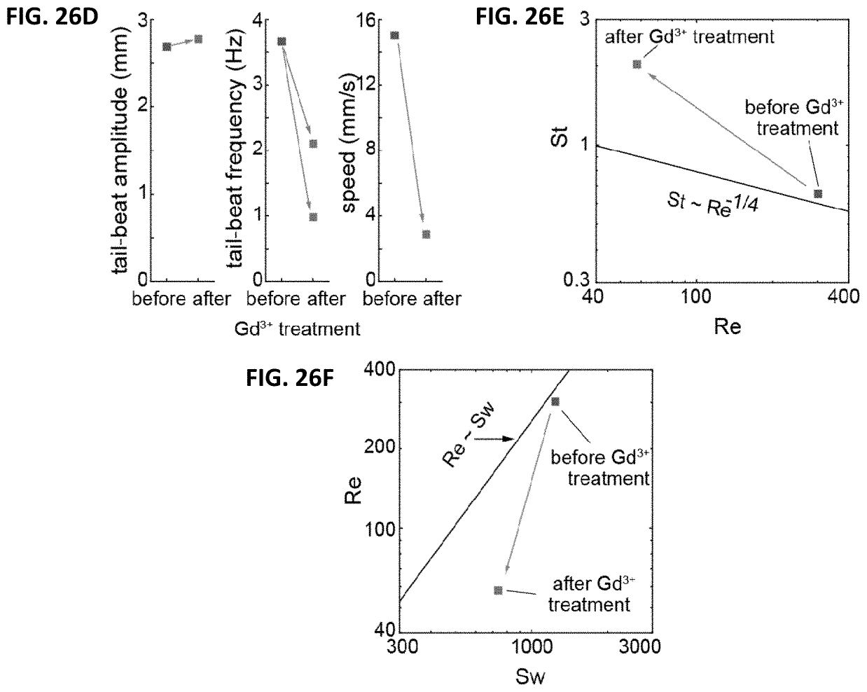

The performance of the biohybrid fish is very sensitive to muscle kinematics and coordination. Some biohybrid fish accelerated by increasing tail-beat amplitude (FIG. 25A and FIG. 25B), which is similar to acceleration in wild-type fish (T. N. Wise et al., J Exp Biol 221, (2018)). This positive relationship between swimming speed and tail-beat amplitude during accelerative locomotion contrasts with the constant tail-beat amplitude regardless of swimming speed during steady locomotion (FIG. 16B). Although St, Sw, and Re numbers increase with its swimming speed (FIGS. 26B and 26C) in the accelerative locomotion, the biohybrid fish exhibited a considerable decrease in propulsive efficiency as speed increases as shown by deviation from optimal St-Re and Re-Sw relationships of aquatic swimmers (FIGS. 26B and 26C). The inhibition of muscle coordination with a stretch-activated ion channel blocker, Gd³⁺, also led to a drastic reduction of 80.8% in Re and 40.6% in Sw and the deviation from optimal St-Re and Re-Sw relationships of aquatic swimmers (FIGS. 19D-19F, FIGS. 26D-26F), which demonstrate that muscular coordination is necessary to achieve effective and efficient swimming.

### G. Long-Term Performance of the Biohybrid Fish

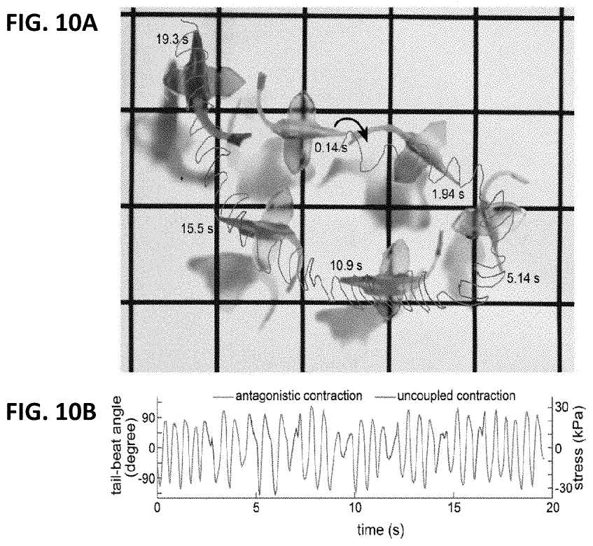

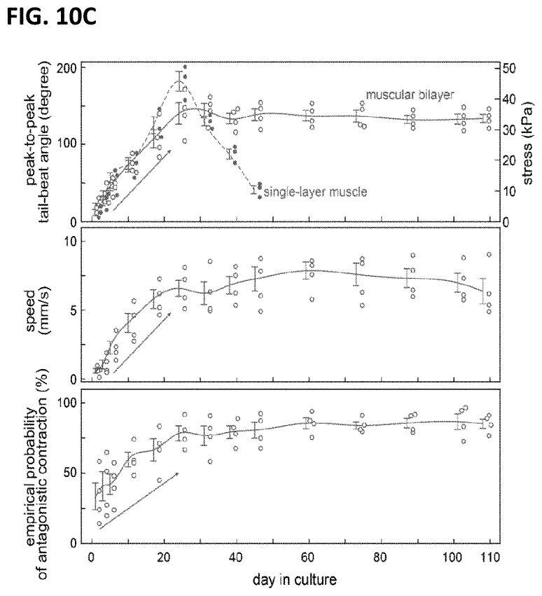

Given the autonomous antagonistic muscle contractions of the biohybrid fish, whether this spontaneous activity would improve its long-term performance was analyzed. The biohybrid fish maintained spontaneous activities for 108 days (16-18× over the biohybrid stingray—6 days (S. J. Park et al., Science (New York, N.Y.) 353, 158-162 (2016)) or skeletal muscle-based biohybrid actuator—7 days (Y. Morimoto et al., Science Robotics 3, 10 (2018))), equivalent to 38 million beats (FIGS. 10A-10B) and its locomotion could also be controlled by external optogenetic stimulation throughout this time. The autonomously swimming biohybrid fish also increased muscle contraction amplitude, maximum swimming speed, and muscle coordination for the first month before maintaining its swimming performance over 108 days (FIG. 10C). In contrast, biohybrid fish equipped with a single-layered muscle showed deteriorating tail-beat amplitude within the first month (FIG. 10C). These data demonstrate the potential of muscular bilayer systems and mechano-electrical signaling as a means to promote maturation of in vitro muscle tissues.

### Summary

As described in this example, two functional design features of the heart—mechano-electrical signaling and automaticity—were integrated into a biohybrid platform and an autonomously actuating cardiac muscular system was created in a biohybrid fish. This biohybrid fish is a closed-loop system where the muscle contraction-induced bending is used as a feedback input to the endogenous mechanosensors—stretch-activated ion channels in the muscles. These channels respond to this feedback input and induce muscle activation and contraction, producing self-sustainable rhythmic BCF propulsion. The self-driven spontaneous contractions in the muscular bilayer induced coordinated global tissue-level contractions with comparable efficiencies to wild-type fish. Alternatively, integrated optogenetic control enabled overriding internal control mechanisms to stop and control asynchronous muscle contractions. There are few, if any, closed-loop mechanical fish robots that are free-swimming, and fish robots typically require numerous actuators and sensors to control fin movements that are difficult to engineer at smaller size scale (mm to cm scale) (R. Du, Z. Li et al., Eds. (Springer Berlin Heidelberg, Berlin, Heidelberg, 2015), pp. 1-24). However, by integrating the cardiac activation system as an embedded mechanism of both sensing and control enabled the generation of fish-like locomotion at the smaller size scales (G. V. Lauder et al., Eds. (Springer Berlin Heidelberg, Berlin, Heidelberg, 2015), pp. 25-49). The use of biological muscle actuators with intrinsic closed-loop control simplifies the construction compared with current mechanical robotic systems and provides control beyond existing biohybrid systems.

Additionally, the muscular bilayer construct of the Example provides a platform for studying tissue-level cardiac biophysics. It demonstrated that dynamic axial stretching can induce excitations and contractions on a beat-by-beat basis in engineered human stem cells-derived CMs tissues by contributing to antagonistic muscle contractions. It was found that antagonistic contractions are streptomycin and Gd³⁺-sensitive, which indicates that mechano-electrical signaling via stretch-activated ion channels is one of the essential mechanisms that mediate antagonistic contractions. Interestingly, in normal myocardium where CMs are mechanically and electrically coupled, mechano-electrical signaling contributes to synchronizing local ventricular repolarization and protects against cell-to-cell repolarizations and contractile heterogeneities across the heart (T. Opthof et al., Cardiovascular research 108, 181-187 (2015)). In contrast, in the muscular bilayer where antagonistic muscle pairs are mechanically coupled yet electrically decoupled across sides, mechano-electrical signaling generated stretch-induced depolarizations on a beat-by-beat basis. The stretch-induced excitations and contractions were also observed in quiescent single CMs and in a resting ventricular myocardium (T. A. Quinn et al., Circulation. Arrhythmia and electrophysiology 10, (2017)), but these observations were restricted to the ectopic responses of CMs to acute mechanical stimulation, which induced reentrant arrhythmias. The muscular bilayer construct of the Example is the first to demonstrate that the mechano-electrical signaling of CMs could induce self-sustaining muscle excitations and contractions for extended periods (108 days, equivalent to 38 million beats). These findings are aligned with the growing appreciation for cardiac stretch-activated channels and mechano-electrical signaling mechanism as targets of heart rhythm management (T. A. Quinn et al., Circulation. Arrhythmia and electrophysiology 10, (2017); T. A. Quinn et al., Cardiovascular research 108, 1-3 (2015)). The longevity of the autonomously moving fish system also raises the question of whether a feedback between repetitive electrical and mechanical activity and the regulation of its molecular elements via altered gene expression or other basic cellular processes is correlated.

The G-node, an isolated cluster of cells connected through a single conducting exit pathway, initiated spontaneous activation waves by reducing the impedance between source and sink. G-node integration improved locomotion speeds by enhancing the pacing frequency. This increased frequency in the presence of the G-node is reminiscent of entrainment in re-entry cycles where the focus shortens the re-entry cycle (J. Almendral et al., Pacing Clin Electrophysiol 36, 508-532 (2013)). Another possible underlying mechanism of the increased frequency is that the G-node produced regular contractions and consequently induced stronger and more rapid contractions of the muscular bilayer, which could enhance the dynamics of antagonistic, asynchronous muscle contractions. The G-node functionality as a node of automaticity in the biohybrid fish indicates that functionally, a pacemaker may be defined by its geometry and source-sink relationships as well as its ion channel expression.

Taken together, the technology described here represents foundational work for the goal of creating autonomous systems capable of homeostatic regulation and adaptive behavioral control. More importantly, the results suggest an opportunity to revisit long standing assumptions of how the heart works in biomimetic systems that allow a more granular analysis of structure-function relationship in cardiovascular physiology.

### Materials and Methods

The following Materials and Methods were used in the Example.

### Human Stem Cell-Derived Biohybrid Fish Fabrication

Fabrication of the tissue-engineered fish (FIG. 13) built upon the processes derived from gelatin-based muscular thin films with modifications (S. J. Park et al., Circulation 140, 390-404 (2019); M. L. McCain et al., Biomaterials 35, 5462-5471 (2014)). First, the paper body of biohybrid fish was fabricated. Two pieces of laboratory tape (General-Purpose Laboratory Labeling Tape, VWR, Radnor, Pa.) were attached together on their adhesive side (FIG. 13A). The biohybrid fish outline (FIGS. 14A-14C) was then laser cut (Model 24, Epilog Laser, Golden, Colo.) onto the laboratory tape (FIG. 13B). Fish body areas where gelatin would be added were also cut out, and placed in 1:1 bleach/deionized water solution for 30 min and in 70% ethanol solution for 30 min to remove any remaining residue.

The laser-cut laboratory tape was placed on the polydimethylsiloxane (PDMS, Sylgard 184, Dow Corning, Midland, Mich.) stamp with line groove features (25 μm ridge width, 4 μm groove width, and 5 μm groove depth) to make a micropatterned gelatin body parallel to the biohybrid fish's longitudinal axis (FIGS. 13C-13E). 20% w/v gelatin (Sigma Aldrich, St. Louis, Mo.) and 8% w/v microbial transglutaminase (MTG, Ajinomoto, Fort Lee, N.J.) with phosphate-buffered saline (PBS, Invitrogen, Carlsbad, Calif.) were warmed to 65° C. and 37° C., respectively for 30 minutes. Then, the solutions were mixed to produce a final solution of 10% w/v gelatin and 4% w/v MTG. 500 μl of the gelatin mixture was quickly pipetted onto the inner fish body areas of the laser-cut laboratory tape. Another PDMS stamp with same line groove features was placed on the MTG and gelatin mixture and then a 500 g scale calibration weight was placed on the PDMS stamp to make thin gelatin body. A glass barrier was placed around the samples to prevent the gelatin from fully drying. The gelatin portions were cured and crosslinked overnight at room temperature. After the gelatin cured, the weight was carefully removed along with excess gelatin on the sides of the stamp. To minimize damage to the micro-molded gelatin, the coverslip and stamp were immersed in distilled water to re-hydrate the gelatin for an hour. The stamp was then carefully peeled off the gelatin. The resulting paper and gelatin body of biohybrid fish was sterilized in 70% ethanol for 30 min and UV treated for 2 min. Within the custom culturing acrylic chip, the biohybrid fish substrate is rinsed with PBS. An EDC (1-ethyl-3-(3-dimethylaminopropyl)carbodiimide hydrochloride, Thermo Fisher Scientific Inc, MA), N-hydroxysulfosuccinimide (sulfo-NHS, Thermo Fisher Scientific Inc, MA), and fibronectin (FN, BD Biosciences, Sparks, Md.) crosslinking to gelatin procedure is then followed. A 10 μl of 0.4 mg/ml EDC and a 10 μl of 1.1 mg/ml sulfo-NHS in sodium acetate buffer (pH 5.5) solution mixed into a 100 μl of 1 mg/ml fibronectin. After 15 mins incubation add the entire (120 μl) FN-EDC-NHS solution to 1880 uμl sterile PBS for a final fibronectin concentration of 50 ug/ml. Then add the FN-EDC-NHS solution incubate on the substrate. After 2 hours of incubation with FN-EDC-NHS solution, the same procedure is followed for the other side of the tissue-engineered fish substrate, and incubated in a 37° C. incubator for at least 1 hour (FIG. 13F) before the cell seeding.

Commercial human stem cell-derived CMs (human stem cell-derived CMs, Axiogenesis, Ncardia, Belgium) were transduced with Channelrhodopsin-2 (ChR2) and ChrimsonR lentivirus at a multiplicity of infection of 5 for 24 hours. ChR2-expressing human stem cell-derived CMs were seeded onto one side at first (FIG. 13G) at a density of 220 k/cm². After a day of culture, ChrimsonR-expressing CMs were seeded onto the other side of the biohybrid fish (FIG. 13H). The biohybrid fish were released from the excess laboratory tape after 3 days in culture (FIG. 13I). Lastly, a plastic floater fin (polymethylpentene film, Sigma Aldrich, St. Louis, Mo.) was inserted into the tissue-engineered fish (FIG. 13J).

### Fabrication of Acrylic Chip for Muscular Bilayer Culture and Biohybrid Fish

The chip was designed within the vector graphics editing software CorelDraw (Corel, Canada). 6.35-millimeter-thick acrylic was then laser cut with a CO₂ laser engraver (VersaLaser 2.0, Universal Laser Systems, Scottsdale, Ariz.). Half of the cut outs were made to be bottoms of a whole chip by introducing threads into the cut acrylic. The acrylic chips were placed in a 70% ethanol bath along with screws and placed in a sonicator for sterilization and cleaning.

### Culture of Human Stem Cell-Derived Cardiomyocytes (Human-CMs)

Human stem cell-derived cardiomyocytes (husk-CMs) were purchased from Axiogenesis and Ncardia (Leiden, Netherlands) and cultured using the manufacturer's protocols. Briefly, 500 μl of culture media (Ncardia, Leiden, Netherlands) was added to each frozen vial of cells and thawed at 37° C. Thawed cells were resuspended in 9 mL of complete culture media (Axiogenesis, Ncardia, Leiden, Netherlands) and incubated in a fibronectin (0.01 μg/mL, BD Biosciences, Bedford, Mass.)-coated T25 flask in a 37° C. incubator for 4 hours, after which puromycin was supplemented to a final concentration of 2 μg/ml. The culture media was replaced with fresh media after an additional 24 hours, and then replaced every 48 hours for cell maintenance until use. For cell seeding on the biohybrid fish, human cardiomyocytes were dissociated with 0.25% trypsin-EDTA (Life Technologies, CA) and resuspended in puromycin free media.

### Harvest and Culture of Neonatal Rat Ventricular Myocytes (NRVMs)

Animal procedures were performed under protocols approved by Harvard University's Institutional Animal Care and Use Committee. Neonatal rat ventricular myocytes (NRVMs) (2-day old) were isolated as previously published (A. W. Feinberg et al., Science (New York, N.Y.) 317, 1366-1370 (2007)). Briefly, ventricles were removed from 2-day old Sprague Dawley rat pups (Charles River Laboratories, MA). Then, the manually minced tissues were placed in a 0.1% trypsin (Sigma Aldrich, St. Louis, Mo.) Hanks' Balanced Salt solution (HBSS) solution at 4° C. for approximately 14 hours. For additional enzymatic digestion, a 0.1% type II collagenase (Worthington Biochem, NJ) solution at room temperature was used to isolate ventricular myocytes. NRVMs were further dissociated by centrifuging, resuspending with HBSS, and passing the isolated cell solution through a 40 μm cell strainer. The solution was pre-plated twice for 50 minutes each at 37° C. in a M199 cell media (Life Technologies) supplemented with 10% heat-inactivated FBS (Life Technologies, CA) to remove fibroblasts and endothelial cells. Resulting NRVMs were seeded onto the biohybrid fish in M199 cell media (Life Technologies, CA) supplemented with 10% heat-inactivated FBS (Life Technologies, CA).

### Optogenetics: Plasmid Constructs and Viral Transduction

pLenti-Synapsin-hChR2(H134R)-EYFP-WPRE (E. S. Boyden et al., Nat Neurosci 8, 1263-1268 (2005)) and FCK-ChrimsonR-GFP (N. C. Klapoetke et al., Nature methods 11, 338-346 (2014)) was a gift from Karl Deisseroth (Addgene plasmid #20945; RRID:Addgene_20945) and Edward Boyden (Addgene plasmid #59049; RRID:Addgene_59049), respectively. hChR2 and ChrimsonR lentiviral vectors (cTnT-ChR2-eYFP and cTnT-ChrimsonR-mCherry) were constructed by cloning inserts (hChR2(H134R) and ChrimsonR) to lentiviral vectors with a cardiac-specific promotor (the cardiac troponin T, cTnT) and a fluorescent tag (enhanced yellow fluorescent protein, eYFP or red fluorescent protein, mCherry). The lentivirus was produced and purchased from VectorBuilder Inc (Chicago, Ill.) and used to infect both human-CMs and NRVMs with lentivirus (5×10⁶ TU/ml) per 1 million CMs.

### Optical Stimulation for Independent Activation of Muscles

Light-emitting diode sources (LED) were used to independently activate ChR2 and ChrimsonR transduced cardiac tissues. Fiber-coupled LED light source (Prizmatix, Israel) at 450 nm and 630 nm were mounted through mono fiber optic cannulas (flat end, 400 μm diameter, NA 0.48, Doric Lenses Inc, Canada). To change the pacing frequency and duration, each LED source was independently controlled by analog signals that were synthesized with an analog output module (NI 9264, National Instruments, Austin, Tex.) by a custom software written (Code and scripts. Zenodo (2021); http://doi.org/10.5281/zenodo.5618323/) in LabVIEW (National Instruments, Austin, Tex.), as previously published (S. J. Park et al., Science (New York, N.Y.) 353, 158-162 (2016)). In addition, the analog output module was triggered by digital trigger signals that were generated by two push button switches through a digital board (USB-6501, National Instruments, Austin, Tex.), allowing the digital signals to change the frequency without time-delay, as previously published (S. J. Park et al., Science (New York, N.Y.) 353, 158-162 (2016)).

### Performance Measurement and Analysis of Biohybrid Fish

The biohybrid fish was placed in Tyrode's solution (1.8 mM CaCl₂, 5 mM glucose, 5 mM Hepes, 1 mM MgCl₂, 5.4 mM KCl, 135 mM NaCl, and 0.33 mM NaH₂PO₄ in deionized water, pH 7.4, at 37° C.; Sigma Aldrich, St. Louis, Mo.) in a 22 cm square chamber (VWR International, Radnor, Pa.) on a hotplate (VWR International, Radnor, Pa.) and temperature was maintained at 35° C. to 37° C. The recording of swimming performance of biohybrid fish was filmed with a sCMOS camera (Pco.edge, PCO AG, Germany) coupled with a zoom camera lens (Thorlabs Inc, Newton, N.J.) at 50 frames per second or a single-lens recorder (AXP55 4K Handycam, Sony, Japan) with a 26.8 mm wide-angle lens at 50 frames per second.

For the longevity measurements, the biohybrid fish were detached after 3 days in culture and incubated in complete culture medium (Ncardia, Leiden, Netherlands), allowing them for self-paced swimming. Every one or two weeks, the performance measurement was conducted in a 37° C. Tyrode's physiological salt solution. After each measurement, the biohybrid fish were placed back and incubated in complete culture medium. The culture medium was exchanged with 12 ml of fresh medium for each biohybrid fish every 4 days for cell maintenance.

To check the effect of mechanical signaling on the antagonistic contraction of muscular bilayer tissues, the biohybrid fish were treated with 250 μM streptomycin or 100 μM Gd³⁺ (both from Sigma-Aldrich, St. Louis, Mo.) before recording, streptomycin or Gd³⁺ for at least 24 hours.

Digital videos recorded during locomotion experiments were converted to image stacks using a custom-made Matlab program (24) (R2020b, Mathworks, Natick, Mass.). The head, body, and tail positions of the biohybrid fish were tracked using an image processing software (ImageJ, NIH, Bethesda, Md.). The moving distance of the biohybrid fish was measured during each tail-beat cycle, and the swimming speed was calculated by dividing the cumulative distance travelled by the total time. Tail-beat amplitude was measured as the distance between maximum right and left excursions. Tail-beat angle (θ) was calculated as the body angle difference between chord lines of its anterior body and caudal fin at the maximum right and left excursions. Antagonistic contraction was determined by checking whether a muscle contraction induced the subsequent contraction of the opposite side muscle with any delay or not. The empirical probability of antagonistic contraction was determined by dividing the number of antagonistic contractions by the total number of contractions.

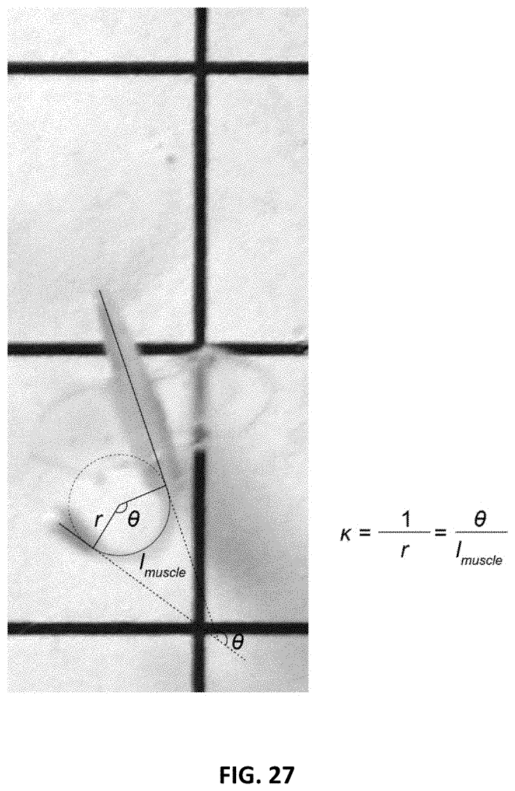

The stress of the muscular bilayer was estimated by considering the geometric relationship of the curvature (κ), radius of curvature (r), length of muscle tissue (l_{muscle}), and tail-beat angle (θ) (κ=1/r=θ/l_{muscle}) (FIG. 27) and using a modified Stoney's equation for anisotropic tissue (S. J. Park et al., Science (New York, N.Y.) 353, 158-162 (2016)):

$$σ = \frac{{E}_{gelatin} {t}_{gelatin}^{2}}{6 {t}_{muscle} ( 1 - {v}_{gelatin}^{2} )} κ = \frac{{E}_{gelatin} {t}_{gelatin}^{2}}{6 {t}_{muscle} ( 1 - {v}_{gelatin}^{2} )} \frac{θ}{{l}_{muscle}} ,$$

where t_{muscle} is thickness of muscle tissue, t_{gelatin} is thickness of gelatin, E_{gelatin} is young's modulus of gelatin and υ_{gelatin} is Poisson's ratio of gelatin (Y. Akiyama et al., PloS one 7, e38274 (2012)).

### Optical Mapping of Muscle Circuit

Calcium activities of the muscle circuit were monitored with a calcium indicator, X-Rhod-1 (Invitrogen, Carlsbad, Calif.), using a modified tandem-lens macroscope as previously published (S. J. Park et al., Science (New York, N.Y.) 353, 158-162 (2016); S. J. Park et al., Circulation 140, 390-404 (2019)). Briefly, tandem-lens macroscope (Scimedia, Costa Mesa, Calif.) used in this example was equipped with a high-speed camera (MiCAM Ultima, Scimedia, Costa Mesa, Calif.), a plan APO 1× objective, a collimator (Lumencor, Beaverton, Oreg.) and a 200-mW mercury lamp for epifluorescence illumination (X-Cite exacte, Lumen Dynamics, Canada). A filter set (excitation filter: 580/14 nm, dichroic mirror: 593 nm cut-off, emission filter: 641/75, Semrock, Rochester, N.Y.) was used for X-Rhod-1 imaging.

The biohybrid fish was incubated with 2 μM X-Rhod-1 for 30 min at 37° C., rinsed with culture medium with 2% FBS to remove nonspecifically associated dye, and incubated again for 30 mins for complete de-esterification of the dye. Then, the biohybrid fish was rinsed with Tyrode's solution and mounted on a 37° C. heating stage (Warner Instruments, Hamden, Conn.) of the tandem-lens macroscope.

Post-processing of the raw calcium data was conducted with custom software written in MATLAB (24) (R2020b, MathWorks, Natick, Mass.). A spatial filter with 3×3 pixels was applied to improve the signal-noise ratio. Activation time of each pixel was calculated at the average maximum upstroke slope of multiple pulses of X-Rhod-1 signals over a 5 second recording window. The total activation time was determined as the difference between activation times at the last and the first activation sites along the posterior body of biohybrid fish.

### Particle Imaging Velocimetry of Biohybrid and Animal Fishes

Flow fields generated by the biohybrid and animal fish were monitored using Particle imaging velocimetry (PIV).

For the PIV measurement of biohybrid fish, algae particles (10-12 μm diameter, Tetraselmis sp., Reed Mariculture) were seeded in Tyrode's solution in a water chamber. 450 nm and 630 nm LED sources were used to independently activate ChR2 and ChrimsonR transduced cardiac tissues through mono fiber optic cannulas (flat end, 400 μm diameter, NA 0.48, Doric Lenses Inc, Canada). While infrared LED sources (IR30, CMVision, Houston, Tex.) illuminated the particles, the motions of the particles were recorded at 100 frames per second with a sCMOS camera (Pco.edge, PCO AG, Germany).

For the PIV measurement of freely-swimming animal fish, individual larval zebrafish, white molly, and Micro-devario kubotai were placed in a recirculating flow tank at speeds of 1.5 to 3.2 body lengths per second (not including zebrafish: for zebrafish, flow was not circulated). While infrared LED sources (IR30, CMVision, Houston, Tex.) illuminated seeded algae particles, the motions of the particles were recorded at 250-500 frames per second with a high-speed video camera (Photron PCI-2014, San Diego, Calif.).

Flow patterns of both biohybrid and animal fish were analyzed by calculating the velocity distribution within seeded particles between successive video frames using an open-source software (PIVlab 2.02 (W. Thielicke et al., Journal of Open Research Software 2, e30 (2014))) written in MATLAB (R2014b, MathWorks, Natick, Mass.).

### Immunofluorescent Staining and Imaging of Biohybrid Fish

Biohybrid fish was fixed for 12 min in 4% paraformaldehyde (Electron Microscopy Sciences, Hatfield, Pa.) and 0.5% Triton-X (Sigma-Aldrich, St. Louis, Mo.) in PBS at 37° C. and incubated with mouse anti-sarcomeric α-actinin monoclonal primary antibody (Sigma-Aldrich, St. Louis, Mo.), and then stained with DAPI (Sigma-Aldrich) and goat anti-mouse secondary antibody conjugated to Alexa-647 (Invitrogen, Carlsbad, Calif.). The biohybrid were mounted on glass slides with ProLong Gold antifade mountant (Invitrogen, Carlsbad, Calif.). All images of biohybrid fish were acquired with an inverted microscope (Olympus IX83, Tokyo, Japan) with an attached spinning disk confocal system (Andor, Concord, Mass.), and three-dimensional reconstructions of images of muscular bilayer were analyzed and processed using image-processing software (ImageJ 1.52p, NIH, Bethesda, Md.).

### Muscle Mass Measurement of Biohybrid Fish

The muscle mass of 42-days old biohybrid fish was measured by a laboratory scale (Mettler Toledo, Columbus, Ohio). The muscle mass was measured by subtracting the mass of gelatin-paper substrate (without cell seeding) from the total mass of biohybrid fish. In another way, the muscle mass of biohybrid fish using the single cell mass and the number of seeded cardiomyocytes on the gelatin portions were estimated. To estimate the mass of single cardiomyocyte, the mass of two million of cardiomyocytes was measured and then divided by the number of cardiomyocytes.

### Local Source-Sink Mismatch in Various Corner Designs

In cardiomyocytes, pacemaking arises from an interplay between hyperpolarizing and dominating depolarizing currents during the phase 4 depolarization period (the period between repolarization and the rising phase of the subsequent action potential). In the sinoatrial node, the hyperpolarization-induced inward current (HCN isoforms) of cardiac pacemaker cells plays a major role in pacemaking (W. Liang et al., Stem Cells. 38, 352-368 (2020)). However, in the case of G-node of this example where stem cell-derived CMs and NRVMs supposedly lack the expression of HCNs, the pacemaking potentials are a result of inward currents produced by Ca²⁺ cycling (driven by rhythmic releases of intracellular Ca²⁺ from the sarco/endoplasmic reticulum).

The remaining question was how a region of cells initiate coordinated pacemaking and how this relates to electrical cell-to-cell coupling. The geometrical node design played a crucial role here because the current being exchanged between individual cells of different membrane potentials is locally accumulated in the membrane capacitance at the edges and is reflected at the tissue edges (A. G. Kléber et al., Physiol Rev 84, 431-488 (2004); A. G. Kléber et al., Biophysics Reviews 2, 031301 (2021)). The reflection of intracellular currents at the tissue edges synchronizes the spontaneous activity in the structurally isolated small tissues like a G-node and increases their firing rate. The mechanism of reflection at the corners of cultures behave similarly (since downstream impedance is reduced), in particular the anterior corners with acute angles albeit less than in the G-node, and as a result, firing is enhanced in the whole anterior side (AD, AV and G-node).

To test the theoretical considerations, the role of geometric factors of the G-node and sink that determine the leading muscle activation site in human stem cell-derived cardiomyocyte tissue (FIGS. 21-23) were investigated.

1) G-node integration: G-node integrated tissues predominantly activated the muscle construct at the G-node compared to the four corners of the muscle tissue (G-node: 72% vs corners: 5-9% in FIG. 21).

2) Sink's corner design: Rounding the sink's corners decreased the corners' activation probability, thus increasing the probability of activation at the G-node activation to 91% (A3 in FIG. 21).

3) G-node's corner design: To investigate the contribution of a sharp corner to the probability of initial activation at the G-node, square and diamond G-nodes were compared to the circular G-node design of this example. The square and diamond G-nodes (B2 and B3 in FIG. 22) have a similar probability of activation at the G-node to the circular design (83 and 87% vs. 91%), which indicates that acute angles in a small source tissue like the G-node is not critical in determining the activation site. Rather, the effect of the borders of the small G-node to enhance the electrical interaction between the cells (through so-called reflection of electronic current) produces a relatively fast and synchronized activation (A. G. Kléber et al., Y. Physiol Rev 84, 431-488 (2004); A. G. Kléber et al., Biophysics Reviews 2, 031301 (2021)).

The contribution of the corner designs in the tissue to the probability of initial activation at the corner was quantified. First, the number of downstream cells to be activated by a spontaneously activated cell at the corner was estimated: the number of cells in the given area was calculated. An area 1-mm away from the corner was chosen, because a 1-mm radius corner in the A3 design (FIG. 21) significantly decreased the probability of initial activation at the corner. Next, given that cells in a corner compete with cells at the G-node to initiate activation waves, a variable ζ, comparing the two probabilities of initial activation at the corner (P) and at the G-node (P_{G-node}), was introduced,

ζ=P/P_{G-node}.

ζ of various G-node-integrated tissue designs (FIG. 23, designs A2, A3, and biohybrid fish) were calculated and the number of downstream cells was compared to the ζ of various designs (FIG. 23, cell density is 150,000/cm²).

### Histology of Animal Fish

All animal procedures were done in accordance with the guidelines of Harvard University's Animal Care and Use Committee. Fishes were anesthetized in neutrally buffered MS222. Fish were either bred in lab or purchased from Uncle Ned's Fish Factory (Millis, Mass.).

Tissue samples of fishes were washed by PBS and then fixed in 4% Paraformaldehyde (SigmaAldrich, St. Louis, Mo.), which were incubated at 4° C. for 24 hours. The samples were decalcificated with Decalcifier-Original (Avantik, Pine Brook, N.J.) for 2.5 h and incubated with 5% sodium sulfate solution for 12 h. Decalcification, paraffin embedding, sectioning, imaging, and staining with Masson Trichrome stain were completed by Applied Pathology Systems (Shrewbury, Mass.).

### Statistical Analysis

Statistical analysis was performed using JMP Pro 15 (SAS Institute, Inc.). Functional performances of the biohybrid fish were compared using one-way ANOVA followed by Tukey-Kramer honestly significant difference test. Data represent mean±SEM. The numbers of fish indicate the numbers of independent experiments.

### EQUIVALENTS

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

As may be recognized by those of ordinary skill in the pertinent art based on the teachings herein, numerous changes and modifications may be made to the above-described and other embodiments of the present disclosure without departing from the spirit of the invention as defined in the appended claims. Accordingly, this detailed description of embodiments is to be taken in an illustrative, as opposed to a limiting, sense.

Those skilled in the art will recognize, or be able to ascertain using no more than routine experimentation, many equivalents to the specific embodiments of the invention described herein. Such equivalents are intended to be encompassed by the following claims.

1. An engineered cardiac tissue structure comprising:
a support having a first side; and
a first engineered cardiac tissue layer disposed at the first side of the support and including a first plurality of cardiomyocytes, the first engineered cardiac tissue layer including a first region having a first area, a second region having a second area smaller than the first area, and a third region forming a conduction pathway for propagation of action potentials between the second region and the first region, the second region forming a geometrically insulated cardiac tissue node.

2. The engineered cardiac tissue structure of claim 1, wherein a minimum width of the third region as measured perpendicular to the conduction pathway, is less than a maximum width of the second region.

3. (canceled)

4. The engineered cardiac tissue structure of claim 1, wherein the first region, the second region and the third region collectively form a cardiac cell region having a cardiac cell region perimeter, and wherein the first plurality of cardiomyocytes do not contact any cardiac cells beyond the cardiac cell region perimeter.

5. The engineered cardiac tissue structure of claim 4, wherein the cardiac cell region perimeter around the first region has one or more corners, and wherein the cardiac cell region perimeter is rounded at each of the one or more corners.

6. The engineered cardiac tissue structure of claim 1, wherein the first region, the second region, and the third region are sized and shaped for spontaneous contraction of the first engineered cardiac tissue layer to initiate in the second region more often than in the first region; or
wherein the first region, second region, and third region are sized and shaped for a higher probability of initiation of spontaneous contraction of the first engineered cardiac tissue layer in the second region than in the first region.

7.-9. (canceled)

10. The engineered cardiac tissue structure of claim 1, wherein the second region acts as a cardiac pacemaker for the engineered cardiac tissue structure.

11. The engineered cardiac tissue structure of claim 1, further comprising:
a second engineered cardiac tissue layer disposed at a second side of the support opposite the first side of the support, the second engineered cardiac tissue layer having an anisotropic tissue orientation and including a second plurality of cardiomyocytes, the first engineered cardiac tissue layer physically separated from the second engineered cardiac tissue layer by a thickness of the support.

12. The engineered cardiac tissue structure of claim 11, wherein excitation and contraction of the first engineered cardiac tissue layer bends the support and strains the second engineered cardiac tissue layer inducing excitation and contraction of the second engineered cardiac tissue layer, and wherein the excitation and contraction of the second engineered cardiac tissue layer bends the support and strains the first engineered cardiac tissue layer inducing excitation and contraction of the first engineered cardiac tissue layer thereby producing antagonistic cyclic contractions.

13. The engineered cardiac tissue structure of claim 12, wherein the antagonistic cyclic contractions are self-sustaining cyclic contractions.

14. (canceled)

15. A self-propelled swimming structure comprising:
the engineered cardiac tissue structure of claim 12;
a front body portion coupled to or attached to a first end of the engineered cardiac tissue structure; and
a rear body portion coupled to or attached to a second end of the engineered cardiac tissue structure.

16.-18. (canceled)

19. An engineered cardiac tissue structure comprising:
a support having a first side and a second side opposite the first side, the first side being patterned to promote anisotropic tissue formation, and the second side being patterned to promote anisotropic tissue formation;
a first engineered cardiac tissue layer disposed at the first side of the support, the first engineered cardiac tissue layer having an anisotropic tissue orientation and including a first plurality of cardiomyocytes; and
a second engineered cardiac tissue layer disposed at the second side of the support, the second engineered cardiac tissue layer having an anisotropic tissue orientation and including a second plurality of cardiomyocytes, the first engineered cardiac tissue layer physically separated from the second engineered cardiac tissue layer by a thickness of the support;
wherein excitation and contraction of the first engineered cardiac tissue layer bends the support and strains the second engineered cardiac tissue layer inducing excitation and contraction of the second engineered cardiac tissue layer, and wherein the excitation and contraction of the second engineered cardiac tissue layer bends the support and strains the first engineered cardiac tissue layer inducing excitation and contraction of the first engineered cardiac tissue layer thereby producing antagonistic cyclic contractions.

20. The engineered cardiac tissue structure of claim 19, wherein the antagonistic cyclic contractions are self-sustaining.

21. The engineered cardiac tissue structure of claim 20, wherein the antagonistic cyclic contractions are spontaneous.

22.-30. (canceled)

31. A method of forming a functional cardiac tissue structure, the method comprising:
providing or obtaining a support having a first side and a second side opposite the first side, the first side being patterned and configured to promote anisotropic tissue formation, and the second side being patterned and configured to promote anisotropic tissue formation;
seeding the first side of the support and the second side of the support with cardiomyocytes; and
growing a first cardiac tissue layer on the first side of the support and growing a second cardiac tissue layer on the second side of the support, the first cardiac tissue layer physically separated from the second cardiac tissue layer by a thickness of the support, thereby forming a functional cardiac tissue structure.

32. The method of claim 31, wherein during growth of the first cardiac tissue layer on the first side of the support and growth of the second cardiac tissue layer on the second side of the support, excitation and contraction of the first cardiac tissue layer bends the support and strains the second cardiac tissue layer inducing excitation and contraction of the second cardiac tissue layer, and the excitation and contraction of the second cardiac tissue layer bends the support and strains the first cardiac tissue layer inducing excitation and contraction of the first cardiac tissue layer producing antagonistic cyclic contractions.

33.-34. (canceled)

35. A method for identifying a compound that modulates a cardiac tissue function, the method comprising:
providing the engineered cardiac tissue structure of claim 1;
contacting the engineered cardiac tissue structure with a test compound; and
determining the effect of the test compound on a cardiac tissue function in the presence and absence of the test compound, wherein a modulation of the cardiac tissue function in the presence of the test compound as compared to the cardiac tissue function in the absence of the test compound indicates that the test compound modulates the cardiac tissue function, thereby identifying a compound that modulates a cardiac tissue function.

36. A method for identifying a compound useful for treating or preventing a cardiac tissue disease, comprising
providing the engineered cardiac tissue structure of claim 1;
contacting the engineered cardiac tissue structure with a test compound; and
determining the effect of the test compound on a cardiac tissue function in the presence and absence of the test compound, wherein a modulation of the cardiac tissue function in the presence of the test compound as compared to the cardiac tissue function in the absence of the test compound indicates that the test compound modulates the cardiac tissue function, thereby identifying a compound useful for treating or preventing a cardiac tissue disease.

37. The method of claim 35, wherein the cardiac tissue function is a biomechanical activity.

38. The method of claim 37, wherein the biomechanical activity is one or more of contractility, cell stress, cell swelling, and rigidity.

39. The method of claim 37, wherein the biomechanical activity is one or more of stem cell activation, stem cell maturation, tissue morphogenesis, and tissue remodeling.

40.-41. (canceled)
